(19) United States
(12) Patent Application Publication
(10) Pub. No.: US 2024/0271071 A1
(43) Pub. Date: Aug. 15, 2024

(54) METHOD AND APPARATUS FOR FORMING A MICROFLUIDIC GEL STRUCTURE

(71) Applicant: Mimetas B.V., Oegstgeest (NL)

(72) Inventors: Claudia Al-Mardini, Oegstgeest (NL); Todd Peter Burton, Oegstgeest (NL); Sebastiaan Johannes Trietsch, Oegstgeest (NL)

(21) Appl. No.: 18/568,418

(22) PCT Filed: Jun. 8, 2022
(86) PCT No.: PCT/EP2022/065506

§ 371 Date: Dec. 8, 2023

(30) Foreign Application Priority Data
Jun. 10, 2021 (NL) 2028424

(51) Int. Cl. C12M 3/06; B01L 3/00

(52) U.S. Cl. CPC C12M 23/16; B01L 3/502707; B01L 2200/027; B01L 2200/0689; B01L 2200/12; B01L 2300/12; B01L 2400/0457

(57) ABSTRACT

A method for creating a lumenized gel structure is described. The method comprises introducing a first liquid comprising a gel precursor solution into a microfluidic network, the microfluidic network comprising a capillary pressure barrier at a position generally defining a boundary between first and second regions of the microfluidic network; allowing the first liquid to enter the first region of the microfluidic network and align itself along the capillary pressure barrier, thereby forming a liquid-air meniscus of the first liquid at the boundary between the first and second regions of the microfluidic network; forming a lumen through the first liquid by contacting the first liquid with a second liquid, wherein the second liquid has a viscosity which is lower than the viscosity of the first liquid; and allowing or causing the first liquid to gelate to form a gel structure comprising a lumen therethrough. Also described is an apparatus comprising a gel with a lumen extending therethrough, and uses of the apparatus and the lumenized gel structure in assays.

### TECHNICAL FIELD OF THE INVENTION

The present invention relates to a method and apparatus that enable 3D culture of cells, allowing for a controlled and reliable vascularization and/or perfusion of organoid assays and/or cell cultures. It equally relates to uses for investigating cellular responses to stimulants resulting from the apparatus and methods.

### BACKGROUND OF THE INVENTION

In a drive towards emulating ever more physiologically relevant conditions in cell culture, numerous models have been developed to enable, for example, perfusion flow, and co-culture in pre-clinical cell-based models for assessing drug efficacy and/or ADME safety.

Microfluidics has become a popular platform technology for such in vitro cell culture models due to the inherent flow of liquids or media during use, along with advances in microengineering techniques that facilitate and enable fabrication of complex microfluidic networks. However, there is still much interest in generating models that simulate or reproduce the cellular environment in the different organs of the human or animal body.

Organoid culture, or more generally speaking 3D cell culture, can be performed in a variety of manners. 3D spheroids can be formed in so-called hanging drop plates (see for instance WO 2010/031194) or low adhesion microtiter plates. Although it is claimed that these spheroids have significantly improved predictivity to standard cell cultures, it is not used for most organoid cultures. The reason is that organoids typically require an extracellular matrix component, such as Matrigel, or collagen that is not present in the hanging drop or low adhesion plate spheroids. Parallel efforts led to the development of 3D cell-culture models in which cells are grown embedded in an extracellular matrix. This approach enhances expression of differentiated functions and improves tissue organization (Pampaloni et al. (2007). Nat Rev Mol Cell Biol 8: 839-84).

Typical platforms to grow organoids comprise standard petri dishes, microtiter plates and in some cases Transwell® plates from Corning. In these cases the organoids are grown in an extracellular matrix (ECM) or on an ECM coated well. As already addressed above these organoids lack the presence of vasculature, thus limiting their growth as beyond a certain size hypoxic and in a later stage necrotic cores may be formed. Also it is hypothesised that the presence of endothelium is crucial for the development towards a physiological relevant tissue, as the endothelium excretes important factors for the target tissue.

Microfluidic cell culturing is an increasingly important technology, finding use in drug screening, tissue culturing, toxicity screening, and biologic research.

Numerous microfluidic systems, devices, methods and manufacturing techniques are known, including those disclosed in patent documents such as WO 2008/079320, WO 2013/151616, WO 2010/086179, WO2012/120101, or as commercially available from, for example, Mimetas, Leiden, The Netherlands (e.g. OrganoPlate; www.mimetas.com). While no particular limitations should be read from those applications and documents into any claims presented herein, these documents provide useful background material.

In A Novel Dynamic Neonatal Blood-Brain Barrier on a Chip. S. Deosarkar, B. Prabhakarpandian, B. Wang, J. B. Sheffield, B. Krynska, M. Kiani. PLOS ONE, 2015 a microfluidic device was developed to generate vasculature that used a sieving like structure to separate the endothelium from astrocytes in an attempt to generate a blood-brain barrier type structure. In WO 2007/008609 A2 a similar sieve like structure is used to form cell aggregates in order to create a tissue morphology that is better mimicking e.g. the physiology of a liver.

There remains a need for methods and apparatuses that allows perfused culture of ECM supported biological tissues that more closely resemble the in vivo situation. Of particular interest are methods and apparatuses that enable patterning of multiple cell types in adjacent constructs, while minimizing the spatial separation and physical obstruction between such constructs. The technology should be also compatible with current-day readout and handling equipment.

It has been an object of the present invention to address some or all of the above mentioned needs.

### SUMMARY OF THE INVENTION

According to a first aspect of the invention there is provided a method for creating a lumenized gel structure, comprising:
introducing a first liquid comprising a gel precursor solution into a microfluidic network, the microfluidic network comprising a capillary pressure barrier at a position generally defining a boundary between first and second regions of the microfluidic network;
allowing the first liquid to enter the first region of the microfluidic network and align itself along the capillary pressure barrier, thereby forming a liquid-air meniscus of the first liquid at the boundary between the first and second regions of the microfluidic network;
forming a lumen through the first liquid by contacting the first liquid with a second liquid, wherein the second liquid has a viscosity which is lower than the viscosity of the first liquid; and
allowing or causing the first liquid to gelate to form a gel structure comprising a lumen therethrough.

According to a second aspect of the invention there is provided an apparatus, comprising:
a microfluidic network, the microfluidic network comprising at least two inlets;
   a capillary pressure barrier at a position defining a boundary between first and second regions of the microfluidic network; and
   a gel provided in the first region extending between two inlets of the at least two inlets and confined to the first region by the capillary pressure barrier;
   wherein the gel comprises a lumen extending therethrough between the two inlets of the at least two inlets; the gel having a first surface facing the lumen, a second surface facing the second region of the microfluidic network, and a thickness of the gel between the first surface and the second surface that is 200 μm or less.

According to a third aspect there is provided a use of a lumenized gel structure as produced by the method of the first aspect in an assay, for example an assay selected from one or more of: a barrier function assay, a trans-epithelial electrical resistance (TEER) assay, an immune cell adhesion assay, an immune cell transmigration assay, a transporter assay, and a vasodilation or vasoconstriction assay.

According to a fourth aspect of the invention there is provided a use of an apparatus as defined in the second aspect in an assay, for example an assay selected from one or more of: a barrier function assay, a trans-epithelial electrical resistance (TEER) assay, an immune cell adhesion assay, an immune cell transmigration assay, a transporter assay, and a vasodilation or vasoconstriction assay.

Other preferred embodiments are defined in the description and dependent claims which follow.

The present inventors have unexpectedly found that it is possible to form, in a first region of a microfluidic network, a lumened gel structure having an exposed surface facing another region of the microfluidic network through the strategic positioning of a capillary pressure barrier in combination with viscous fingering techniques. To date, lumened gel structures in microfluidic networks have either filled a microfluidic channel and contacted the channel walls on all sides or have been supported by a membrane in order to allow diffusion into and out of the gel. Since viscous fingering techniques rely on one liquid forming a lumen through another liquid, it is unexpected that the viscous fingering lumenization did not disrupt the surface tension of the first liquid pinned at the capillary pressure barrier which would have led to collapse of the pinned meniscus. While one would expect the need for supporting walls to prevent the lumenizing liquid from flowing out towards the side, this invention shows an unexpected method that allows lumen formation in very close proximity to an open space without constraining walls.

The methods and apparatuses of the present invention enable formation of a 3D constituted tissue in an extracellular matrix having a thin interstitial space, thus mimicking the in vivo situation in a way that has not before been achievable without the use of membranes. This enables controlled co-culture of endothelialized lumen(s) in close proximity to monolayers and/or three-dimensional cultures of tissue-specific cells in a physiologically realistic environment, for example co-culture of endothelium, pericytes, astrocytes and neurons in a configuration and in a matrix that is more realistic than existing microfluidic blood-brain barrier models.

### Definitions

Various terms relating to the apparatus, methods, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

As used herein, the “a,” “an,” and “the” singular forms also include plural referents unless the content clearly dictates otherwise. Thus, for example, reference to “a cell” includes a combination of two or more cells, and the like.

As used herein, “about” and “approximately”: these terms, when referring to a measurable value such as an amount, a temporal duration, and the like, are meant to encompass variations of ±20% or ±10%, more preferably +5%, even more preferably +1%, and still more preferably ±0.1% from the specified value, as such variations are appropriate to perform the disclosed methods.

As used herein, “comprising” is construed as being inclusive and open ended, and not exclusive. Specifically, the term and variations thereof mean the specified features, steps or components are included. These terms are not to be interpreted to exclude the presence of other features, steps or components.

As used herein, “exemplary” means “serving as an example, instance, or illustration,” and should not be construed as excluding other configurations disclosed herein.

As used herein, the term “microfluidic network” refers to one or more channels on or through a layer of material that is covered by a top-substrate or cover, with at least one of the dimensions of length, width or height being in the low (for example less than 5 mm or less than 2 mm) or sub-millimeter range. It will be understood that the term encompasses channels which are linear channels, as well as channels which are branched, or have bends or corners within their path. A microfluidic network typically comprises at least one inlet for administering a volume of liquid, but may comprise multiple inlets for administering volumes of liquid to different regions of the microfluidic network. The volume enclosed by a microfluidic network is typically in the microliter or sub-microliter range. A microfluidic network typically comprises a base, which may be the top surface of an underlying material, at least two side walls, and a ceiling, which may be the lower surface of a top substrate overlying the microfluidic network, with any configuration of inlets, outlets and/or vents as required. The base, side walls and ceiling may each be referred to as an inner surface of the microfluidic network, and collectively may be referred to as the inner surfaces. In some examples, the microfluidic network may have a circular or semi-circular cross-section, which would then be considered to have one or two inner surfaces respectively.

As used herein, the term “capillary pressure barrier” refers to features of an apparatus that keep a liquid-air meniscus pinned at a certain position by capillary forces. A capillary pressure barrier can be considered to divide a microfluidic network having a volume V₀ into two regions or sub-volumes V₁ and V₂ into which different fluids can be introduced. Put differently, a capillary pressure barrier generally defines a boundary between first and second regions of the microfluidic network. It will be understood that while the capillary pressure barrier generally defines a boundary between regions of a microfluidic network, a resultant pinned meniscus of a liquid in one region may not be pinned at the exact location of the capillary pressure barrier, and may stretch or bulge beyond the capillary pressure barrier into an adjacent region while still being pinned. For example, a liquid meniscus may be convex in shape, and be pinned by a capillary pressure barrier, with the convex liquid front extending beyond the footprint of the capillary pressure barrier. The liquid meniscus may also be concave, with the solvent front pinned by the capillary pressure barrier and stretching beyond the footprint of the capillary pressure barrier on the surface of the microfluidic network opposite the capillary pressure barrier.

As used herein, a “linear” capillary pressure barrier is not to be construed as being limited to a straight line. Instead, it is to be construed as having a configuration with two ends, but which may comprise one or more bends or angles. A linear capillary pressure barrier typically intersects at each end with a sidewall of the microfluidic network.

As used herein, the term “endothelial cells” refers to cells of endothelial origin, or cells that are differentiated into a state in which they express markers identifying the cell as an endothelial cell.

As used herein, the term “epithelial cells” refers to cells of epithelial origin, or cells that are differentiated into a state in which they express markers identifying the cell as an epithelial cell.

As used herein, the term “biological tissue” refers to a collection of identical, similar or different types of functionally interconnected cells that are to be cultured and/or assayed in the methods described herein. The cells may be in or take the form of a cell aggregate, a tubular structure or a monolayer. The biological tissue may be constituted of multiple subtypes of cells. For example, the term “biological tissue” encompasses cells derived from or comprise cell lines, organoids, tissue biopsies, tumor tissue, resected tissue material, and embryonic bodies.

As used herein, the term “cell aggregate” refers to a 3D cluster of cells in contrast with surface attached cells that typically grow in monolayers. 3D clusters of cells are typically associated with a more in-vivo like situation. In contrast, surface attached cells may be strongly influenced by the properties of the substrate and may undergo de-differentiation or undergo transition to other cell types.

As used herein, the term “organoid” refers to a miniature form of a tissue that is generated in vitro and exhibits endogenous three-dimensional organ architecture.

As used herein, the term “co-culture” refers to two or more different cell types being cultured in an apparatus described herein. The different cell types can be cultured in the same region of the apparatus (e.g., first region or second region) and/or in different regions (e.g., one cell type in a first region and another cell type in a second region).

For example, an apparatus as described herein may have endothelial cells grown as a tubule having an open lumen in the first region, organ-specific (parenchymal) cells in a second region, separated by a thin layer of gel of the first region. In some examples, an apparatus may comprise at least one lumened gel structure lined with endothelium in the first region and tissue specific cells in the second region. The tissue specific cells may be disposed throughout a gel structure in the second region, lining the second region to form a tubule in contact with the gel structure of the first region (comprising the endothelium-lined lumen); or lining a lumen extending through a gel structure in the second region.

As used herein, the term “lumenized gel structure” refers to a biocompatible gel, more preferably a biologically relevant gel, for example an extracellular matrix, having a lumen running through the gel, enabling formation of, for example, a microvessel having apical and basal surfaces. It should be understood that “lumenized” and “lumened” can be used interchangeably as having the same meaning.

As used herein, the term “lumened cellular component” refers to a biological tissue (i.e. constituted of cells) having a lumen, for example a microvessel having apical and basal surfaces.

As used herein, the term “transplant” or “transplantation” refers to the transfer of tissue, for example tissue explant, or cell aggregates from one location to another, for example from a storage container to a cell culture apparatus.

As used herein, and unless otherwise stated, references to viscosity are to dynamic viscosity and are determined as described by Kane et al (AIP Advances 8, 125332 (2018)). The relationship between the lumen formation and viscosity is understood by a person skilled in the art as Saffman-Taylor instability and described by Saffman and Taylor in 1958 (Proceedings of the Royal Society of London. Series A. Mathematical and Physical Sciences. 245 (1242): 312-329.). The relationship between microchannel dimensions and other relevant properties in relationship to extracellular matrices are e.g. described in Bischel et al (Journal of Laboratory Automation 17(2) 96-103).

### BRIEF DESCRIPTION OF THE FIGURES

The present invention will now be described by way of example only, with reference to the Figures, in which:

FIG. 1A shows cross-sectional views of a sequence of steps in accordance with a method described herein for the formation of a lumenized gel structure;

FIG. 1B shows plan views of a sequence of steps in accordance with a method described herein for the formation of a lumenized gel structure;

FIG. 1C shows cross-sectional views of a sequence of steps in accordance with a method described herein for the formation of a lumenized gel structure;

FIG. 2 shows cross-sectional views of a sequence of steps in accordance with a method described herein for the formation of a lumenized gel structure;

FIG. 3 shows cross-sectional views of a sequence of steps in accordance with a method described herein for the formation of a lumenized gel structure;

FIG. 4A shows cross-sectional views of a sequence of steps in accordance with a method described herein for the formation of a lumenized gel structure;

FIG. 4B shows cross-sectional views of a sequence of steps in accordance with a method described herein for the formation of a lumenized gel structure;

FIG. 5 shows cross-sectional views of an apparatus comprising two adjacent lumened cellular components and remodelling of an extracellular matrix;

FIG. 6A shows a cross-sectional view of an exemplary apparatus of the present disclosure comprising lumened cellular components;

FIG. 6B shows a cross-sectional view of an exemplary apparatus of the present disclosure utilising a lumenized gel structure to form a lumened cellular component;

FIG. 7 shows a cross-sectional view of an exemplary apparatus of the present disclosure comprising three lumened cellular components;

FIG. 8 shows a cross-sectional view of an exemplary apparatus of the present disclosure comprising three lumened cellular components;

FIG. 9A shows an experimentally obtained confocal microscopy cross-sectional view of an apparatus according to FIG. 8;

FIG. 9B shows an experimentally obtained confocal microscopy plan view of an apparatus according to FIG. 5;

FIG. 10 shows an experimentally obtained confocal microscopy cross-sectional view of a further apparatus according to the present disclosure;

FIG. 11A shows a cross-sectional view of a further exemplary apparatus of the present disclosure comprising two lumened cellular components;

FIG. 11B shows an experimentally obtained phase contrast microscopy plan view of an apparatus according to FIG. 11A;

FIG. 12 shows an experimentally obtained phase contrast microscopy image in plan view of the apparatus of FIG. 8 comprising three adjacent lumened cellular components;

FIG. 13 shows an experimentally obtained high resolution image of a plan view of a further apparatus according to the present disclosure;

FIG. 14 shows an experimentally obtained phase contrast microscopy cross-sectional view of a blood-brain barrier model created according to the present disclosure;

FIG. 15 shows an experimentally obtained confocal microscopy cross-sectional view of a blood-brain barrier model created according to the present disclosure;

FIG. 16A shows a cross-sectional view of an embodiment of the apparatus of FIG. 7 comprising one lumened cellular component.

FIG. 16B shows an experimentally obtained phase contrast microscopy plan view of an apparatus according to FIG. 16A.

FIG. 17A shows a cross-sectional view of an embodiment of the apparatus of FIG. 7 comprising two lumened cellular components.

FIG. 17B shows an experimentally obtained phase contrast microscopy plan view of an apparatus according to FIG. 17A

FIGS. 18A, 18B and 18C show experimentally obtained confocal microscopy cross-sectional views of a coronary artery model created according to the present disclosure;

FIG. 19 shows an experimentally obtained phase contrast microscopy cross-sectional view of a T-cell migration model created according to the present disclosure;

FIG. 20 shows an experimentally obtained phase contrast microscopy cross-sectional view of a comparative T-cell migration model; and

FIG. 21 presents quantitative data obtained from the models shown in FIGS. 19 and 20.

### DETAILED DESCRIPTION OF THE INVENTION

### Apparatus

An apparatus is described. The apparatus may be a microfluidic apparatus, also termed herein a microfluidic device. The apparatus comprises at least one microfluidic network, for example a plurality of microfluidic networks. The microfluidic device is preferably in a multi-array format/multi-well format to enable its use in in vitro cell-based assays, pharmaceutical screening assays, toxicity assays, and the like; in particular in a high-throughput screening format. Such multi-well culture plates are available in 6-, 12-, 24-, 48-, 96-, 384- and 1536 sample wells arranged in a rectangular matrix, wherein in the context of the present invention a multi-array configuration of microfluidic networks as herein described are present in the microfluidic device. In one example, the microfluidic device is compatible with one or more dimensions of the standard ANSI/SLAS microtiter plate format. In an alternative embodiment the microfluidic device is in a multi-array format with dimensions of a microscope glass slide. In some examples, the microfluidic device is provided with one or more functions including one or more electrodes for conducting electrical experiments; transparent materials, windows or other modification to enable optical measurements to be taken, and so on.

The microfluidic device therefore preferably has a plurality of microfluidic networks as herein described. In one example, the plurality of microfluidic networks are fluidly disconnected from each other; in other words, each microfluidic network operates independently of any other microfluidic network present on the microfluidic device. In other examples, as will be described later, the microfluidic networks may be connected by one or more connecting channels.

Generally, the microfluidic device is a microfluidic device that comprises at least a microfluidic network having a microfluidic channel. Different configurations of microfluidic channels or networks are possible within the metes and bounds of the invention but may include for example one or more regions for receiving and confining a gel, for example an extracellular matrix.

The microfluidic device generally comprises a microfluidic network, which will now be described in detail.

### Microfluidic Network

The microfluidic network of the microfluidic device generally comprises a base, a microfluidic channel or microfluidic layer and a cover, also referred to herein as a cover layer, and can be fabricated in a variety of manners.

The base, also referred to herein as the base layer, or bottom substrate, is preferably formed from a substantially rigid material, such as glass or plastic, and serves to provide a supporting surface for the rest of the microfluidic network. In one example, the base is of the same or similar dimensions to the well area of a standard ANSI/SLAS microtiter plate.

The microfluidic device or network comprises a microfluidic channel or microfluidic layer disposed on the base. In some examples, the microfluidic network may comprise or be divided into different regions, for example by the presence of a capillary pressure barrier as described herein. In some examples, the microfluidic network may comprise a first region and a second region. A capillary pressure barrier may generally define a boundary between adjacent regions of the network, for example between a first region and a second region. In some examples, the microfluidic network comprises one or more microfluidic channels, each forming a region of the microfluidic network and being served by its own dedicated inlet(s) and/or outlet(s).

The first region and the second region can each have a width dimension between about 100 μm and about 10 mm, or between about 200 μm and about 500 μm, or between about 300 μm and about 400 μm.

A typical method of fabrication of a microfluidic network is to cast a mouldable material such as polydimethylsiloxane onto a mould, so imprinting the microfluidic network into the silicon rubber material thereby forming a microfluidic layer. The rubber material with the network or channel embedded is subsequently placed on a base layer of glass or of the same material to thus create a seal. Alternatively, the channel structure could be etched in a material such as glass or silicon, followed by bonding to a top or bottom substrate (also referred to herein as a cover layer and base layer). Injection moulding or embossing of plastics followed by bonding is another manner to fabricate the microfluidic channel network. Yet another technique for fabricating the microfluidic channel network is by photo lithographically patterning the microfluidic channel network in a photopatternable polymer, such as SU-8 or various other dry film or liquid photoresists, followed by a bonding step. When referred to bonding it is meant the closure of the channel by a cover or base. Bonding techniques include anodic bonding, covalent bonding, solvent bonding, adhesive bonding, and thermal bonding amongst others.

As deduced from the various fabrication methods above, the microfluidic layer may comprise a sub-layer comprising a microfluidic channel disposed on the base layer, or is patterned in either the cover or base layer. In an in use orientation, the microfluidic sub-layer is disposed on the top surface of the base layer. The microfluidic channel may be formed as a channel through a sub-layer of material disposed on the base layer. In one example, the material of the sub-layer is a polymer placed on the base layer and into which the microfluidic channel is patterned. In some examples, the microfluidic layer comprises two or more microfluidic networks, which may be in fluidic communication with each other.

The microfluidic channel may be provided with one or more fluid inlets, and one or more outlets or vents, as required for any particular use of the microfluidic network of the microfluidic device. In order to allow filling, emptying and perfusion of a fluid through the microfluidic network, the microfluidic channel is preferably provided with at least two inlets. In one example, each of the at least two inlets is preferably an aperture in the cover layer. It will be understood that there typically is no geometrical distinction between an in- and outlet and that in many cases they can be used as in- or outlet interchangeably. In particular, it will be understood that an inlet aperture fluidically connected to a region opposite to an inlet aperture through which a liquid is introduced will at that time function as an outlet (or vent), to allow air or excess liquid to be expelled.

The microfluidic network can comprise at least two inlets, each of which is configured to enable introduction of a liquid into a first region of the microfluidic network, or removal of the liquid from the first region. In some examples, the microfluidic network can comprise at least three inlets, for example at least four inlets, with at least two of the inlets being configured to enable introduction of a liquid into a second region of the microfluidic network, or removal of the liquid or expulsion of air from the second region. In some examples, the microfluidic network can comprise yet two further inlets, each configured to enable introduction of a liquid into a third region of the microfluidic network, or removal of the liquid or expulsion of air from the third region. It will be understood that inlets being configured to enable introduction of liquids into any given region of a microfluidic network implies a fluidic connection to that region, for example with fluid interfaces that enable injection or pipetting of a liquid into a region, or into a microfluidic channel that communicates with the region in question. The location of a capillary pressure barrier may indicate the boundary between two different regions of the microfluidic network. It shall be understood that the boundary between two regions is generally aligned with such a capillary pressure barrier in one plane, but that the interface between the two regions can curve away from a projection of the capillary pressure barrier in any direction.

In some examples, the microfluidic device further comprises a top layer disposed on the above-mentioned cover layer, the top layer having one, or at least one well or reservoir in fluidic communication with the rest of the microfluidic device. In some examples, the top layer has a plurality of such wells, and at least one, for example at least two, for example at least three wells are in communication with a microfluidic network or channel of the device. For example, the top layer may comprise a well or reservoir in fluidic communication with a microfluidic network via an inlet aperture provided in a cover layer of the microfluidic network thereby forming a SLAS compliant wellplate. In some examples, the top layer having at least one well and the microfluidic layer are integrally formed. For example, a microfluidic channel may be patterned onto the underside of an injection moulded microtiter plate having at least one well.

### Capillary Pressure Barrier

The microfluidic network of the apparatus comprises a capillary pressure barrier generally defining a boundary of the microfluidic network between a first region of the network and a second region of the network.

The function and patterning of capillary pressure barriers have been previously described, for example in WO 2014/038943 A1. As will become apparent from the exemplary embodiments described hereinafter, the capillary pressure barrier is not to be understood as a wall (or a cavity which can for example be filled with a liquid), but instead consists of or comprises a structure which ensures that such a liquid does not spread due to the surface tension. This concept is referred to as meniscus pinning. As such, stable confinement of a liquid to a region of the microfluidic network can be achieved. In one example, the capillary pressure barrier may be referred to as a confining phaseguide, which is configured to not be overflown during normal use of the cell culture device. The nature of the confinement of a liquid is described herein in connection with the description of the methods of the present invention.

In one example, the capillary pressure barrier is provided on an internal surface of the microfluidic network and comprises a ridge, groove, or line of material that has an increased water-air contact angle with respect to the internal surface of the microfluidic network. In one example, the capillary pressure barrier comprises or consists of a rim or ridge of material protruding from an internal surface of the microfluidic network; or a groove in an internal surface of the microfluidic network. In order to provide a good barrier, the internal angle formed by a sidewall of the rim or ridge and the top of the rim or ridge is preferably less than 110°, for example about 90°, in some examples less than 90°. The same counts for the angle between the sidewall of the ridge and the internal surface of the microfluidic network on which the capillary pressure barrier is located. Similar requirements are placed on a capillary pressure barrier formed as a groove.

An alternative form of capillary pressure barrier is a region of material of different wettability to an internal surface of the microfluidic network, which acts as a spreading stop due to capillary force/surface tension. As a result, the liquid is prevented from flowing beyond the capillary pressure barrier and enables the formation of stably confined volumes in a region of the network. In one example, the internal surfaces of the microfluidic network comprise a hydrophilic material and the capillary pressure barrier is a region of hydrophobic, or less hydrophilic material. In one example, the internal surfaces of the microfluidic network comprise a hydrophobic material and the capillary pressure barrier is a region of hydrophilic, or less hydrophobic material.

Thus, in some examples, the capillary pressure barrier is selected from a rim or ridge, a groove, a hole, or a hydrophobic line of material or combinations thereof. In another embodiment capillary pressure barriers can be created by pillars at selected intervals, the arrangement of which defines a first region or area that is to be occupied by a gel. In one example, the pillars extend the full height of the microfluidic network.

In one example, the capillary pressure barrier is a substantially linear capillary pressure barrier which spans the complete width or length of a microfluidic channel or network and intersects on each end with sidewalls of the microfluidic network.

In another example, the capillary pressure barrier is not linear, but comprises one or more bends or arcuate portions. For instance, the capillary pressure barrier may comprise consecutive angled bends or arcuate portions such that a meandering or even a right-angled shape is created. This way the path of a fluid being guided along the capillary pressure barrier is extended with respect to the path a fluid would follow along a linear capillary pressure barrier. The advantage of having a non-linear capillary pressure barrier is that a lumen created in a fluid aligned along such a capillary pressure barrier can have a non-linear shape, e.g. mimicking the crypt-villi structure of the small intestine, and/or that the length of a non-linear lumen is extended with respect to its linear counterpart.

The intersection of the capillary pressure barrier with a sidewall or more than one sidewall of the microfluidic channel may have an angle on the downstream side of the capillary pressure barrier with respect to the envisioned filling direction of a first fluid that is larger than 70°, more preferably around 90°, more preferably larger than 90°. This angle is preferably as large as possible in order to provide a good barrier, as described in WO 2014/038943.

In some examples, the capillary pressure barrier is patterned on an internal surface of the microfluidic network to mimic a biological structure. Forming the capillary pressure barrier into a shape or configuration to mimic a biological structure facilitates formation of in vitro systems that more closely resemble in vivo systems. For example, the capillary pressure barrier may comprise a sinusoidal shape to mimic crypt villi structures.

### Second Capillary Pressure Barrier

In some examples, the microfluidic network of the apparatus is provided with a second capillary pressure barrier, the form and function of which is substantially as described above. For the avoidance of doubt, references to “a capillary pressure barrier” are to be understood as references to “the first capillary pressure barrier” when a second capillary pressure barrier is present in the microfluidic network.

In some examples, the second capillary pressure barrier defines a boundary between the first region and a third region of the microfluidic network, or a boundary between the second region and a third region of the microfluidic network. Thus, a microfluidic network having two capillary pressure barriers may comprise a first region, a second region and third region, and each of the regions may be served by at least one dedicated fluid interface, for example its own dedicated inlet and outlet, or vent. In some examples, a second capillary pressure barrier is provided at a position generally defining a boundary between the first region and a third region of the microfluidic network; so that a liquid can align itself along the first capillary pressure barrier and the second capillary pressure barrier, thereby forming a third surface of the gel structure facing the third region of the microfluidic network. In some further examples, the second capillary pressure barrier is provided at a position generally defining a boundary between the second region and a third region, optionally allowing the patterning and potentially forming a lumen of a second gel in the third region. It will be understood that multiple capillary pressure barriers, regions and gel structures can be combined to increasingly complex microfluidic networks and gel structures.

### Gel

A gel is provided in a first region of the microfluidic network, extending between two inlets and confined to the first region by the capillary pressure barrier. The gel comprises a lumen extending therethrough between the two inlets and has a first surface facing the lumen, a second surface facing the second region of the microfluidic network, and a thickness of the gel between the first surface and the second surface may be 200 μm or less. In some examples, the lumen is substantially cylindrical, for example having a substantially circular cross-section.

The formation of the lumen through the gel can be performed according to the methods described herein. The combination of the capillary pressure barrier and lumen formation through the gel provides two surfaces of the gel and thus enables or allows formation of a 3D constituted tissue having a thin interstitial space, mimicking the in vivo situation in a way that has not before been achievable.

The gel may be provided in the first region of the microfluidic network by introducing a gel precursor solution into the first region, for example via an inlet that serves the first region, with the lumen being formed through the gel precursor solution according to a method described herein.

The gel or gel precursor includes any hydrogel known in the art suitable for cell culture. Hydrogels used for cell culture can be formed from a vast array of natural and synthetic materials, offering a broad spectrum of mechanical and chemical properties. Suitable hydrogels promote cell function when formed from natural materials and are permissive to cell function when formed from synthetic materials. Natural gels for cell culture are typically formed of proteins and ECM components such as collagen, fibrin, fibrinogen, fibronectin, hyaluronic acid, laminin, or Matrigel, as well as materials derived from other biological sources such as chitosan, alginate or silk fibrils. Since they are derived from natural sources, these gels are inherently biocompatible and bioactive. Permissive synthetic hydrogels can be formed of purely non-natural molecules such as polyethylene glycol) (PEG), poly(vinyl alcohol), and poly(2-hydroxy ethyl methacrylate). PEG hydrogels have been shown to maintain the viability of encapsulated cells and allow for ECM deposition as they degrade, demonstrating that synthetic gels can function as 3D cell culture platforms even without integrin-binding ligands. Such inert gels are highly reproducible, allow for facile tuning of mechanical properties, and are simply processed and manufactured. The gel or gel precursor may comprise a basement membrane extract, human or animal tissue or cell culture-derived extracellular matrices, animal tissue-derived extracellular matrices, synthetic extracellular matrices, hydrogels, collagen, soft agar, egg white and commercially available products such as Matrigel.

Basement membranes, comprising the basal lamina, are thin extracellular matrices which underlie epithelial cells in vivo and are comprised of extracellular matrices, such a protein and proteoglycans. In one example, the basement membranes are composed of collagen IV, laminin, entactin, heparan sulfide proteoglycans and numerous other minor components (Quaranta et al, Curr. Opin. Cell Biol. 6, 674-681, 1994). These components alone as well as the intact basement membranes are biologically active and promote cell adhesion, migration and, in many cases growth and differentiation. Matrigel is an example of a gel based on basement membranes and is very biologically active in vitro as a substratum for epithelial cells.

Many different suitable gels for use in the methods and apparatus described herein are commercially available, and include but are not limited to those comprising Matrigel rgf, BME1, BMElrgf, BME2, BME2rgf, BME3 (all Matrigel variants) Collagen I, Collagen IV, mixtures of Collagen I and IV, or mixtures of Collagen I and IV, and Collagen II and Ill), puramatrix, hydrogels, Cell-Tak™, Collagen I, Collagen IV, Matrigel® Matrix, Fibronectin, Gelatin, Laminin, Osteopontin, Poly-Lysine (PDL, PLL), PDL/LM and PLO/LM, PuraMatrix® or Vitronectin. In one preferred embodiment, the matrix components are obtained as the commercially available Corning® MATRIGEL® Matrix (Corning, NY 14831, USA).

By virtue of the meniscus pinning effect of the capillary pressure barrier and the lumenization of the gel (or precursor), a gel within the microfluidic network (for example in a first region of the network) has two surfaces that can be exposed to air (for example by withdrawing the second liquid from the formed lumen), and/or are accessible for the introduction of other liquids to contact the surface. For convenience, the internal surface of the gel facing the lumen will generally be described as a first surface, and the outer surface of the gel facing an adjacent region of the microfluidic network (for example a second region) will generally be described as a second surface. It will be appreciated that the second surface corresponds to the meniscus of the gel precursor solution that was pinned by the capillary pressure barrier when the gel precursor solution was introduced into the first region, and may therefore extend the length of the capillary pressure barrier. It will be understood that the first surface may extend along the entire length of the microfluidic network, including parts of the network lead up to and away from the capillary pressure barrier and can thus be longer than the second surface.

The gel may have a thickness of 200 μm or less between the first surface and the second surface. It will be understood that the gel thickness may not be uniform along the entire length between the first and second surfaces, meaning that the thickness of 200 μm or less may be a minimum thickness, or a maximum thickness. In some examples, a thickness of the gel between the first surface and the second surface may be 150 μm or less, for example 100 μm or less, for example 50 μm or less, for example 40 μm or less, for example 30 μm or less, for example 20 μm or less, for example 10 μm or less, for example about 1 μm. In some examples, the gel may have a thickness between the first surface and the second surface which is approaching zero, for example a thickness of less than 1 μm, for example less than 500 nm, for example less than 250 nm, for example less than 100 nm, for example less than 50 nm, to replicate an in vivo basal lamina. In some examples, the gel has a thickness between the first surface and the second surface of from 20 nm to 200 μm, for example from 100 nm to 150 μm, for example from 500 nm to 100 μm, for example from 1 μm to 50 μm.

As described above, the gel is present in a first region of the microfluidic network, and may have a surface facing a second region of the microfluidic network, by virtue of the gel being pinned by a capillary pressure barrier. In some examples, the gel is also pinned by a second capillary pressure barrier which defines a boundary between the first region and a third region of the microfluidic network. In these examples, the gel comprises a third surface, facing the third region. It will be appreciated that the third surface, as with the second surface, may by concave, or convex in shape, and may extend or bulge beyond the physical location of the capillary pressure barrier while still being pinned.

In other examples, a second capillary pressure barrier is present, but which defines a boundary between the second region and a third region of the microfluidic network. In these examples, the first region comprising the lumened gel is spaced from the third region by the second region.

In some examples, the microfluidic network comprises an aperture, which may be a different aperture to any inlet aperture used for infilling of a liquid or venting, and the gel structure forms a surface facing and/or substantially sealing the aperture. The aperture may itself function as a capillary pressure barrier, preventing flow of liquid out of the aperture. In some examples, the lumen does not extend to the aperture, and instead extends through the gel underneath the aperture (in the instance when the microfluidic network including the gel is, in use, in a plane below the aperture). The gel structure may therefore have a surface facing and/or substantially sealing the aperture, which can be contacted with a liquid of interest and which can provide access to the gel at a location between the beginning and end of the lumen along its length.

In some examples, the second region also comprises a gel or gel precursor solution, forming a gel-to-gel contact with the gel within the first region. In some examples, the microfluidic network comprises a third region, delineated by a second capillary pressure barrier, and the third region also comprises a gel or gel precursor solution. In these examples, the third region may be adjacent the first region or the second region. Thus, the third region may comprise a gel forming a gel-to-gel contact with the gel within the first region, or a gel forming a gel-to-gel contact with a gel within the second region, or a gel in the first and third region without gel to gel contact between the two. When present, the gel within the second region and the gel within the third region may be lumened gels or gel structures, formed using the methods described herein.

While the present discussion has focussed on the presence of one or two capillary pressure barriers, and the subsequent formation of gel structures within first, second and third regions of the microfluidic network, it will be understood that the disclosure is not limited to such configurations, and that yet further capillary pressure barriers, providing for yet further regions of the microfluidic network, can also be included. Thus, the microfluidic network may generally comprise of N number of regions or lanes and N−1 capillary pressure barriers dividing each region or lane. Thus, the methods described can be used to form a gel in any N lane where a freestanding meniscus is formed, and N lumen may be created for N number of lanes within a microfluidic network.

Once a gel structure within the microfluidic network has been lumenized by a method described herein, one or more cells or cell types can be introduced into the microfluidic network for the purpose of forming, for example gel-supported tubules or vessels, as will be described below in connection with the methods of the disclosure.

### Method for Creating a Lumenized Gel Structure

Described herein is a method for creating a lumenized gel structure, comprising:
introducing a first liquid comprising a gel precursor solution into a microfluidic network, the microfluidic network comprising a capillary pressure barrier at a position generally defining a boundary between first and second regions of the microfluidic network;
allowing the first liquid to enter the first region of the microfluidic network and align itself along the capillary pressure barrier, thereby forming a liquid-air meniscus of the first liquid at the boundary between the first and second regions of the microfluidic network;
forming a lumen through the first liquid by contacting the first liquid with a second liquid, wherein the second liquid has a viscosity which is lower than the viscosity of the first liquid; and
allowing or causing the first liquid to gelate to form a gel structure comprising a lumen therethrough.

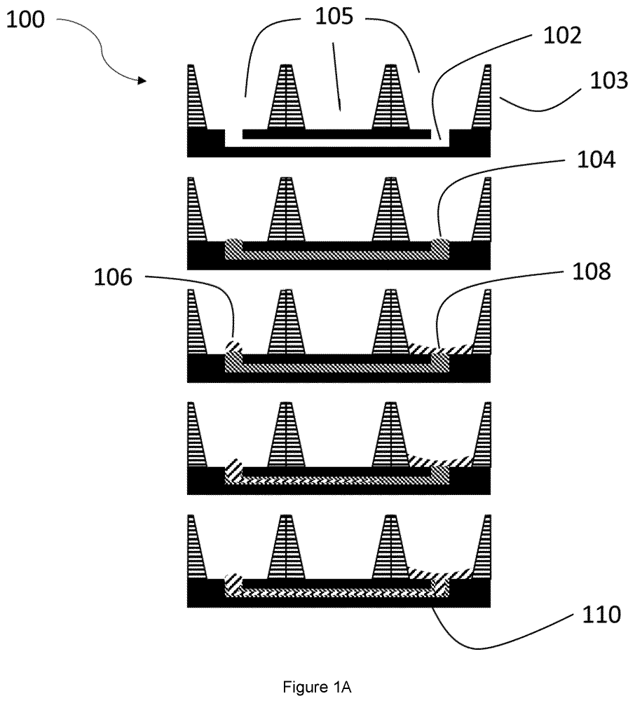

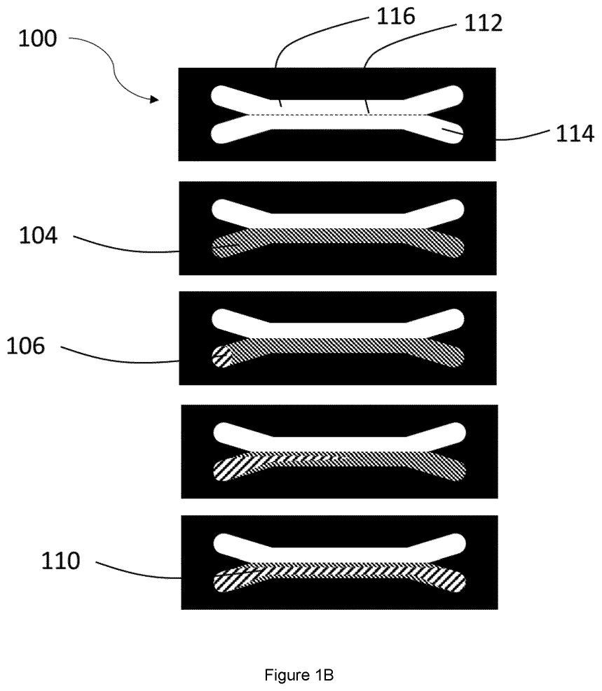

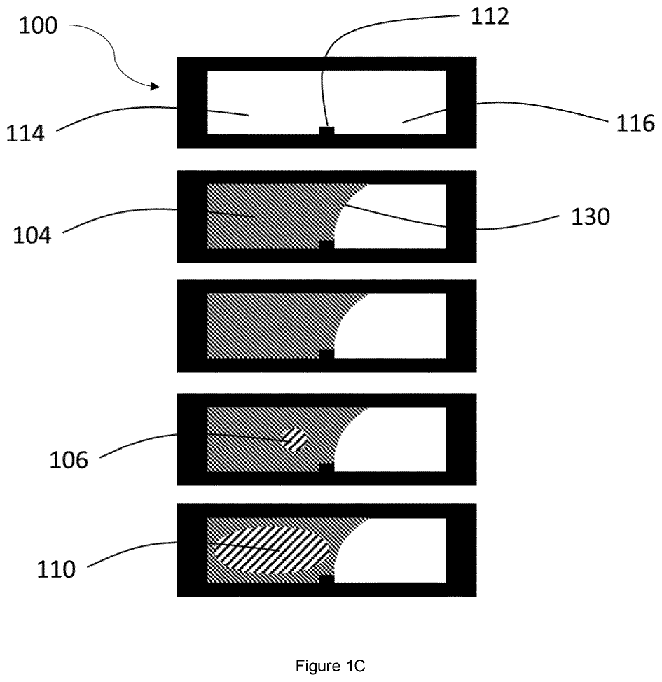

FIGS. 1A, 1B and 1C show a sequence of steps in accordance with a method as described herein. FIG. 1A shows a side-on or cross-sectional view of an apparatus comprising a microfluidic network, while FIG. 1B shows the same apparatus in plan view, and FIG. 1C shows the same apparatus in cross-section perpendicular to the view of FIG. 1A.

Apparatus 100 includes a microfluidic network 102, which in this example is provided with two inlets (not numbered) at either end of the network and accessed from above via a cover layer 103 comprising wells 105.

In one example, the first liquid comprising a gel precursor solution is introduced into the microfluidic network via an inlet providing access to the microfluidic network, for example by injection or insertion. More specifically, the microfluidic network and the inlet are configured so that the inlet communicates with the first region of the microfluidic network, i.e. the inlet and microfluidic network define a flow path from the inlet to the first region. Once the first liquid has been introduced into the microfluidic network, for example via injection, it is allowed to enter the first region, for example via a flow path, and align itself along the capillary pressure barrier. Typically, capillary forces are sufficient to cause the first liquid to flow through the microfluidic network, and no continual injection pressure or back pressure is required. In some examples, a back pressure is applied to allow the first liquid to enter the first region and align itself along the capillary pressure barrier.

The second image of FIGS. 1A to 1C, shows a first liquid 104, comprising a gel precursor solution being introduced into the microfluidic network 102. As can be seen in FIGS. 1B and 1C, a capillary pressure barrier 112 is provided in the microfluidic network 102, which generally defines a boundary between a first region 114 and a second region 116 of the microfluidic network. Capillary pressure barrier 112 is provided as a ridge of material (shown in FIG. 1B as a dashed line) protruding from the floor of microfluidic network 102 into the main body or channel. Once introduced into the microfluidic network 102, first liquid 104 fills first region 114 and aligns itself along capillary pressure barrier 112, forming a liquid-air meniscus running along or parallel to capillary pressure barrier 112, so being generally located at the boundary between the first region 114 and second region 116. Although the meniscus is pinned by capillary pressure barrier 112, it can be seen in FIG. 1C that the part of the meniscus contacting a ceiling of the microfluidic network extends partly into second region 116. However, it will be understood that the liquid-air meniscus of the first liquid is still generally disposed at the boundary, by virtue of the meniscus pinning effect of the capillary pressure barrier 112.

Suitable gels, and precursor solutions include but are not limited to those comprising Matrigel, Matrigel gfr, BME1, BME1gfr, BME2, BME2gfr, BME3 (all Matrigel variants) Collagen I, Collagen IV, mixtures of Collagen I and IV, or mixtures of Collagen I and IV, and Collagen II and Ill), puramatrix, hydrogels, Cell-Tak™, Matrigel® Matrix, Fibronectin, Gelatin, HA, Laminin, Osteopontin, Poly-Lysine (PDL, PLL), PDL/LM and PLO/LM, PuraMatrix® or Vitronectin. In one preferred embodiment, the matrix components to be introduced as a gel precursor solution are obtained as the commercially available Corning® MATRIGEL® Matrix (Corning, NY 14831, USA).

Next, a lumen is formed through the first liquid by contacting the first liquid with a second liquid at a first location in the microfluidic network, wherein the second liquid has a viscosity which is lower than the viscosity of the first liquid. This technique is known as viscous fingering, or the Saffman-Taylor instability, and relies on the different relative viscosities of two liquids for the formation of a lumen. Methods of creating three-dimensional lumen structures in permeable matrices are known in the art (Bischel et al. J Lab Autom. (2012) 17: 96-103; and Bischel et al. Biomaterials (2013) 34: 1471-1477).

A pressure gradient must be created for lumenisation to occur resulting in the displacement of the higher viscosity first liquid by the lower viscosity second liquid. This pressure gradient can be achieved by any number of methodologies, which include, but are not limited to: Laplace force due to the surface tension of a droplet (passive pumping), hydrostatic pressure, pneumatic pressure, mechanical pressure (for example, by means of a syringe pump).

The first liquid, i.e. a gel precursor solution, may have a viscosity that is high enough to form a defined structure but which still allows the second liquid, which is of a lower viscosity, to disperse through the first liquid, e.g., via surface tension-based passive pumping without the need for external pressure-driven flow, and to remove a portion of the first liquid, thereby creating the lumen which extends through or within the first liquid.

In some examples, the first liquid can have a viscosity of about 5 cP to about 10⁶ cP, for example from about 5 cP to 5000 cP, for example from about 30 cP to 1000 cP. The composition and/or viscosity of the second liquid that disperses through the first liquid can vary with the viscosity of the first liquid. In general, the more viscous the first liquid, the higher the viscosity of the second liquid may need to be for it to extend through the first liquid and to create the lumen therethrough. In some examples, the second liquid may have a viscosity of about 0.5 cP to about 5 cP. It shall be appreciated that the absolute value of the viscosity of commonly used hydrogels is dependent on many factors, including dynamic properties such as shear, strain and viscoelastic properties, resulting from the fact that many gel precursors are non-newtonian fluids. Within these ranges however, the gel precursors are always significantly more viscous, e.g. >5 cP, than the fluids used for lumenization such as water or cell culture medium, e.g. approx. 1 cP. As long as the varying and dynamic viscosity of the gel precursor can be expected to always be more viscous than the second fluid, the proposed methods are applicable even if the exact viscosity of the gel is changing or only approximately defined.

Methods of modifying, i.e. the viscosity of gel precursor solutions for forming gel matrices (ECM) are known in the art, and include, for example, adjusting the concentration of the polymer (e.g. collagen) in the solution, where higher concentrations yield higher viscosity; causing a partial gelation of the solution, where more gelation yield higher viscosity; changing the temperature, where lower temperature slows down gelation, but increases viscosity of the ungelled gel precursors, or introducing a viscosity modifier such as a polyethylene glycol into the solution.

The third image in FIGS. 1A to 1C shows a droplet of second liquid 106 introduced onto the top of first liquid 104 filling microfluidic network 102. For the purposes of illustration, second liquid 106 was introduced through the same inlet as first liquid 104, which can be considered the first location. In order for second liquid 106 to push through first liquid 104 and lumenize first liquid 104, a third liquid 108 may be applied to a second location spaced from the first location, e.g. an inlet into microfluidic network 102 which is spaced from the inlet at which second liquid 106 was introduced. Third liquid 108, as second liquid 106, may have a viscosity which is lower than the viscosity of first liquid 104.

Third liquid 108 may be introduced if second liquid 106 is constrained at its inlet due to surface tension at the interface of the inlet. Introducing third liquid 108 can help to break the surface tension, thus requiring a lower pressure of second liquid 106. Third liquid 108 may be of the same or different composition to second liquid 106, and can be, but is not limited to: a liquid selected from HBSS, cell culture media, PBS, TRIS, Water, HEPES, Albumin solution, a balanced salt solution, a second gel or gel precursor solution, or a buffering agent. The third liquid may be identical to the second liquid. The viscosity of the third liquid may be lower, similar or higher than the first liquid. In some examples, second liquid 106 is introduced prior to third liquid 108 being introduced, while in other examples the order of addition is reversed.

In some examples, contacting the gel precursor solution 104 with the second liquid 106 comprises: forming a meniscus of the second liquid 106 that is convex in shape and has a first principal radius of curvature; and contacting the gel precursor solution 104 with the third liquid 108 comprises: forming a meniscus of the third liquid 108 that is concave in shape; or that is convex in shape with a second principal radius of curvature that is smaller than the first principal radius of curvature. Providing a third liquid 108 that forms a meniscus that is concave in shape; or that is convex in shape with a principal radius of curvature that is smaller than the principal radius of curvature of the second liquid 106 that will form the lumen 110 advantageously reduces, or reduces the surface tension of first liquid 104, enabling second liquid 106 to bore through first liquid 104 and form lumen 110.

In this way, a passive pressure-driven flow of the second liquid 106 having a viscosity lower than that of the first liquid gel precursor 104 can pattern a generally circular or elliptical lumen 110 in first liquid 104, as can be seen in the lower images of FIGS. 1A to 1C. In some examples, passive pumping can be facilitated using gravity to apply a pressure difference from one inlet to another inlet, for example by adjusting the inclination of the microfluidic network. In other examples, third liquid 108 is not used, and an external pressure can be applied to force second liquid 106 through first liquid 104.

The dimensions of the lumen can vary with a number of factors, including, but not limited to dimensions of the channels, relative viscosities between the first liquid and the second liquid, volumetric flow rate and/or pressure of the second fluid flowing through the first liquid, and any combination thereof. In some embodiments, a cross-sectional area of the lumen may be over 50% of the cross-sectional area of the channel. In some embodiments, the cross-sectional area of the lumen may be over 90% of the cross-sectional area of the channel, e.g. 98% or 99%. In some examples, the lumen can have a dimension of about 10 μm to about 1000 μm. In some embodiments, the lumen can have a dimension of 20 μm to about 500 μm, for example 50 μm to about 250 μm.

Once first liquid 104 has been lumenized, that is, lumen 110 has been formed therethrough using second liquid 106, gelation, or polymerization, of the gel precursor solution 104 results in a gel structure, for example an extracellular matrix gel structure, comprising lumen 110 extending therethrough.

In some examples, first liquid 104, comprising the gel precursor solution is subjected to a partial gelation prior to lumenization. In other examples, lumenization occurs prior to any gelation of the first liquid 104. Second liquid 106 can be withdrawn from the lumen 110 by application of a positive or negative pressure to any aperture forming an inlet or outlet and can be withdrawn prior to or after gelation has occurred, thus exposing an inner surface of the gel structure. As can be seen in the bottom image of FIG. 1C, the gel structure formed from first liquid 104 has a first surface facing lumen 110, and a second surface facing second region 116, both of which can be modified by addition of one or more cell types, as will be described later.

In some examples, second liquid 106 may itself comprise a gel precursor solution, capable of being lumenized by a boring liquid which has a lower viscosity than the viscosity of second liquid 106. Thus, the methods described may include forming a lumen through the second liquid by contacting the second liquid with a liquid having a viscosity which is lower than the viscosity of the second liquid; and allowing or causing the second liquid to gelate within the first gel structure to form a laminar gel structure comprising a lumen therethrough. By varying the composition of the gel precursor solution of second liquid 106 relative to the composition of the gel precursor solution of first liquid 104, a stratified, potentially concentric, system can be created. In particular, in some examples, one or both of first liquid 104 and second liquid 106 may independently include one or more cell types, for example one or more cells of mesenchymal origin, for example selected from stromal cells, muscle cells, pericytes, fibroblasts, and myofibroblasts.

The above method has been described with reference to a microfluidic network having one capillary pressure barrier, defining two regions of the microfluidic network. However, it will be understood that the methodology can be applied to more complex systems, having two, or more, capillary pressure barriers that result in three, or more, regions of the microfluidic network, each having one or more inlets and being independently patternable with a matrix gel structure, which may or may not be lumened, and/or with one or more cell types that can form organoid bodies, or vasculature which lines the surfaces of the region in question.

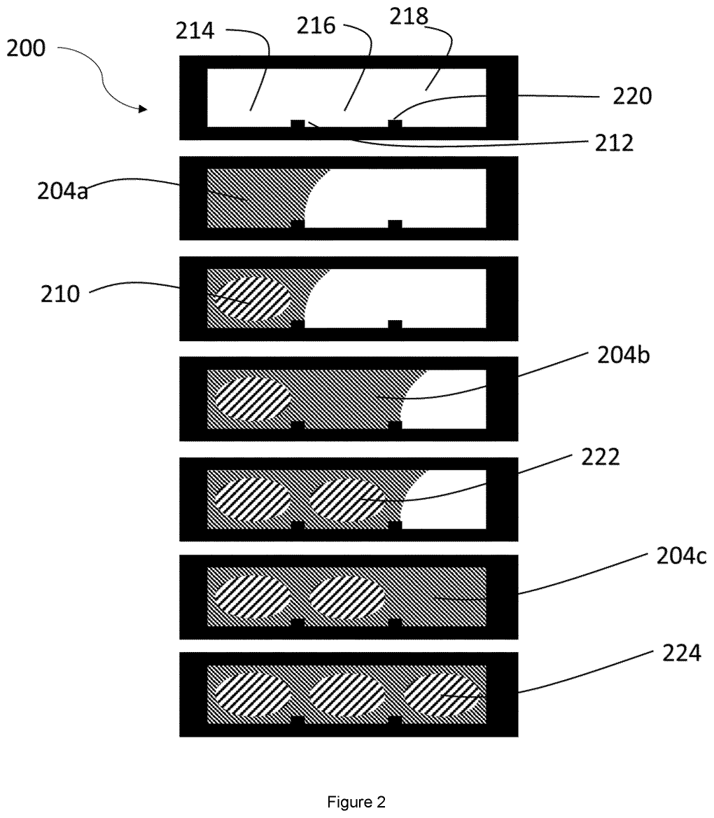

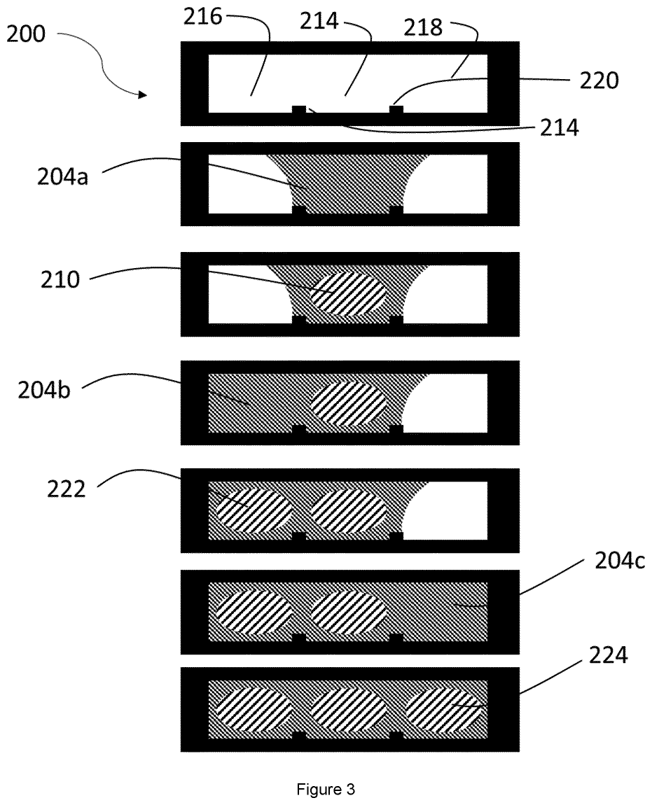

Thus, in some examples, the methods may include introducing a further liquid, for example a second gel precursor solution, into the second region of the microfluidic network and allowing the second gel precursor solution to contact the gel structure along the length of the capillary pressure barrier; forming a lumen through the second gel precursor solution by contacting the second gel precursor solution with a liquid having a viscosity which is lower than the viscosity of the second gel precursor solution; and allowing or causing the second gel precursor solution to gelate, to form a second gel structure comprising a lumen therethrough and contacting the first gel structure, as depicted in FIGS. 2 and 3.

FIGS. 2 and 3 both show an apparatus 200 comprising a microfluidic network having two capillary pressure barriers, indicated at 212 and 220. Capillary pressure barrier 212 and capillary pressure barrier 220 are both provided as ridges which protrude from the floor or base of the microfluidic network into the channel, but it will be appreciated that other configurations of capillary pressure barrier are possible, as described herein.

In FIG. 2, capillary pressure barrier 212 defines a boundary between first region 214 and second region 216. In this example, capillary pressure barrier 220 defines a boundary between second region 216 and third region 218.

The second image of FIG. 2 shows first liquid 204a, a gel precursor solution, being introduced into first region 214, and formation of lumen 210 therethrough is shown in the third image. After gelation of first liquid 204a to form a gel structure, a second gel precursor solution 204b can be introduced into second region 216, which can also be lumenized as described herein using the viscous fingering methodology, to provide a second lumen 222 as seen in the third and fourth images. Following gelation of second gel precursor solution 204b to form a gel structure with a lumen, a third gel precursor solution can be introduced into third region 218, and a third lumen 224 provided.

An alternative sequence of steps is depicted in FIG. 3, resulting in the same arrangement as that of FIG. 2. In FIG. 3, first region 214 can be considered as being in the centre of the microfluidic network, with the boundary between that and second region 216 to the left being capillary pressure barrier 212, and the boundary between first region 214 and third region 218 being capillary pressure barrier 220.

As can be seen in the third image of FIG. 3, the methodology and arrangement of the present disclosure enables formation of a lumenized gel structure having three surfaces: the first surface faces lumen 210, the second surface faces second region 216, while the third surface faces third region 218. While such an arrangement may be advantageous for certain applications, FIG. 3 demonstrates that it is also possible to introduce gel precursor solutions 204b and 204c into second region 216 and third region 218 respectively, and to form lumens 222 and 224 through second region 216 and third region 218 respectively.

While the sequence of steps in FIGS. 2 and 3 shows the filling of all three regions with gel, and lumenization of all three, it will be appreciated that there may be advantages in only forming lumenized gel structures in first region 214 and second region 216, while leaving third region 218 accessible for culture media or test solutions.

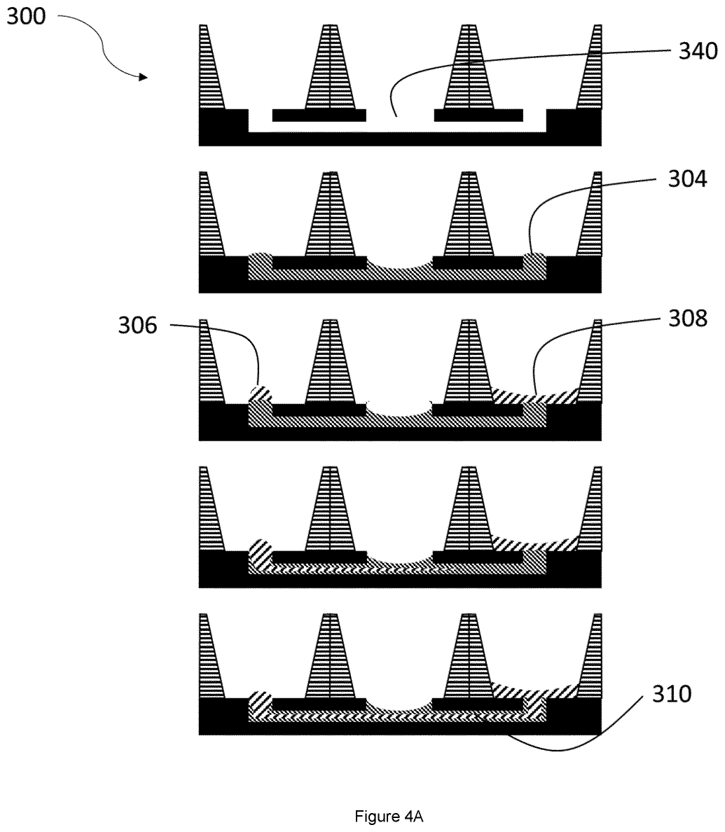

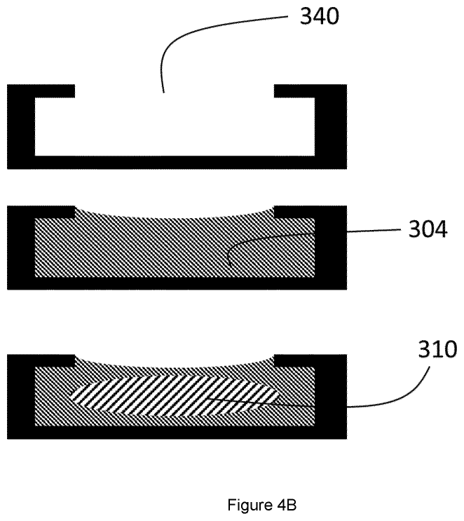

FIGS. 4A and 4B show an alternative apparatus 300, in which an aperture 340 is provided in addition to any dedicated fluid interface inlets and outlets. Aperture 340 may function as a capillary pressure barrier, pinning first liquid 304 so that it forms a meniscus, and surface extending across aperture 340. As with the methods described before, a first liquid gel precursor solution 304 is introduced into the microfluidic network of apparatus 300, and a second liquid 306 and a third liquid 308 are introduced at either ends of the network, to break surface tension and cause lumenization of the first liquid 304.

As can be seen in FIGS. 4A and 4B, lumen 310 extends from first liquid 306 to second liquid 308 and does not extend to aperture 340. It can be seen, therefore, that a given region of the microfluidic network may have a plurality of apertures, which may be considered inlets or outlets, and that lumenization can be controlled so that a lumen extends only between desired locations (apertures) of the microfluidic network. For example, forming the lumen may comprise minimizing a principal radius of curvature at one aperture of the plurality of apertures so as to cause the lumen to extend to that one aperture and only that one aperture. This can be achieved by applying the second liquid to the aperture and minimizing the surface tension at that aperture.

In this way, lumenization can occur below the plane of an aperture, resulting in a further surface of the gel structure. FIG. 13 shows a high-resolution image taken from above such an apparatus, looking through an aperture to a lumen running through a gel below the aperture. The arrow overlaying the image indicates the path of the gel and lumen.

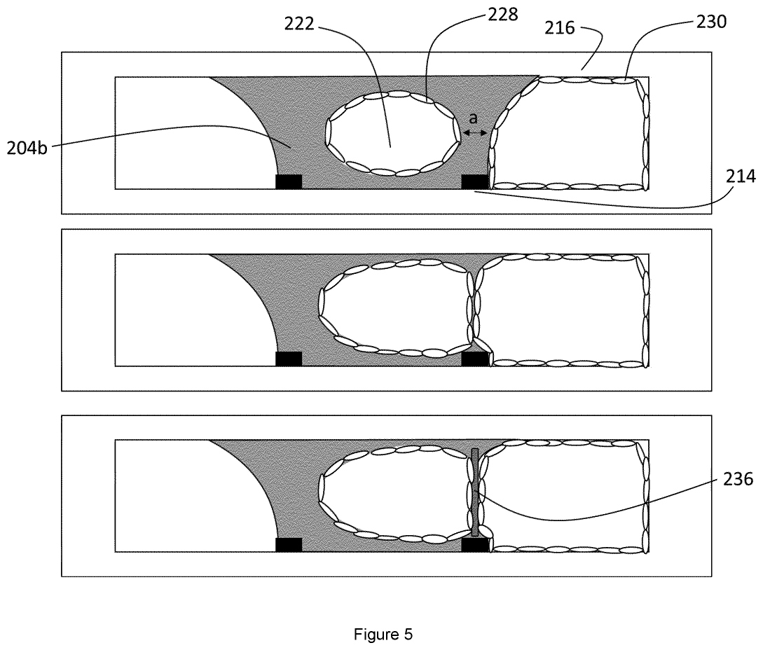

The top image of FIG. 5 depicts an apparatus in which a gel structure formed from a gel precursor 204a is provided in a first region 214 and pinned by capillary pressure barriers. A lumen 222 extends through the gel structure, and is lined with a tubule of cells 228, which may be endothelial or epithelial cells. In second region 216, which is free of a gel structure, a second tubule of cells 230 is present, with an interstitial space between the tubules marked as “a”. Cells 228 and/or cells 230 can be allowed or stimulated to remodel the gel structure by reducing the thickness of the gel structure between the first surface and the second surface and/or by secreting one or more ECM components. Cells can be stimulated by growth factors, glucose and oxygen levels to become more or less metabolically active. More proliferative and more motile cells typically degrade ECMs more actively. Activation of ECM degradation and remodeling enzymes, e.g. MMPs or ADAMTS, by activators such as MMP-3, plasmin, kallikrein, tryptase, furin etc, will further enhance ECM degradation and reducing the thickness of the gel structure. Conversely, ECM remodeling can be inhibited using MMP inhibitors, ROCK inhibitors etc which may conserve thicker gel structures.

As can be seen in the second image of FIG. 5, the gel matrix has been remodeled (in this case degraded) to such an extent that the distance between tubules has been reduced to almost zero, resulting in (almost) direct cell-to-cell contact between neighbouring tubules.

It will be appreciated that any cells present in the microfluidic network, for example cells introduced via a gel precursor solution, or introduced into the lumen of a gel structure (as or via a cellular tubule), or introduced into a region of the microfluidic network faced by the gel structure, may remodel the gel structure.

FIG. 5 illustrates the potential of an apparatus and method described herein for providing an interstitial distance between tubules that can reduce to zero or almost zero, via remodelling of an extracellular matrix gel. Generally, one or more types of cells can be introduced into the microfluidic network, and allowed or stimulated to remodel the gel structure by reducing the thickness of the gel structure. In some examples, the thickness of the gel structure is remodelled between the first surface and the second surface of the gel structure (that is, between the surface facing the lumen and an external surface of the gel structure). Remodelling can comprise deposition or degradation of any ECM component. The one or more cells may secrete one or more ECM components that result in remodelling.

Degradation and remodelling of the extracellular matrix gel can promote cell movement or migration, for example by orienting matrix components such as collagen fibers. Remodelled ECM, resulting in topographical changes such as a reduced thickness or fiber orientation, is often observed in areas where cancer epithelial cells invade, but a remodelled ECM may also influence the behaviour of stromal, endothelial or immune cells in the local environment, and so there is a need to investigate behaviour of cells in remodelling an extracellular matrix, or behaviour of cells responding to a remodelled extracellular matrix. The methods and apparatuses described herein address that need.

In the bottom image of FIG. 5, the cells (in this case epithelial cells) have been allowed to remodel the ECM by secreting glycoproteins and collagen to form a new basal lamina 236 between the tubules. In this way, a more realistic co-culture model for investigation can be obtained and the remodelled ECM may have a thickness between the first surface and the second surface which is approaching zero, for example a thickness of less than 1 μm, for example less than 500 nm, for example less than 250 nm, for example less than 100 nm, for example less than 50 nm.

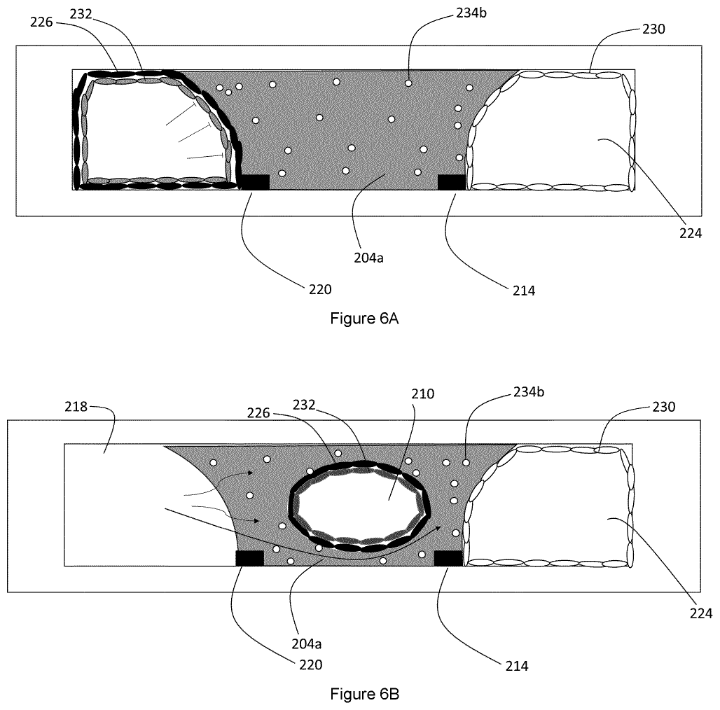

The present invention has particular advantages over non-lumenized gel structures in 3-lane microfluidic devices, which can be explained with reference to FIGS. 6A and 6B. FIG. 6A shows a typical model for investigating molecular transport across an interstitial layer between biological structures in a 3-lane system. In this model, a gel structure formed from a gel precursor solution is provided in the central region of a microfluidic network, pinned by capillary pressure barriers 214 and 220.

The gel structure may include or comprise an extracellular matrix such as collagen or Matrigel®, and may include cells (for example stromal cells) 234b dispersed throughout the gel. To the right side of the gel structure is a monolayer tubule of cells 230 (for example endothelial or epithelial cells) with a lumen 224, while to the left of the gel structure is a bilayer tubule formed of cells 226 and 232, which could, for example be a pericyte/endothelium-lined tubule as described above.

The bilayer and the stromal cells are here introduced as an example. The invention functions equally well for two monolayer tubes and no cells added to the ECM gel.

In this set up, any material (test compound, nutrient media) must be introduced into the microfluidic network via the bilayer tubule or the monolayer tubule, and may not easily reach the adjacent gel structure and/or the other tubule, as it must pass the layer(s) of cells forming the tubule—as can be seen with the blocked arrows within the bilayer tubule formed of cells 226 and 232.

In contrast, if the bilayer tubule is formed in a lumened gel structure as in FIG. 6B, then one lane of the 3-lane system (for example third region 218 in FIG. 6B) can be kept for adding a trigger, drug, staining reagent, marker or reporter molecule or can be used for sampling of e.g. excreted metabolites and basally excreted cytokines. Since the gel structure is pinned by capillary pressure barrier 220 without requiring any membrane, a surface of the gel faces third region 218 and can be contacted by any solution introduced into third region 218, thereby facilitating exchange and transport of material through the interstitial gel structure to the tubules so that there is free exchange of metabolites, nutrients, compounds, drugs, triggers, chemokines, cytokines and oxygen between the fluid in the third region and the gel structure and basal side of the tubules.

It goes without saying that a person skilled in the art would recognize that cells in an extracellular matrix environment (i.e. the gel structure) are still motile through the gel network. Motility is enabled by cell matrix interaction through focal adhesions such as integrins, actin remodelling, by collagenases and many other mechanisms that enable motility of cells in an extracellular matrix environment. Cells might also migrate in and out from an ECM gel during the course of experimentation and/or across an epithelial or endothelial barrier. This is all enabled by the lumened component in the gel structure and the free surface of the gel facing third region 218.

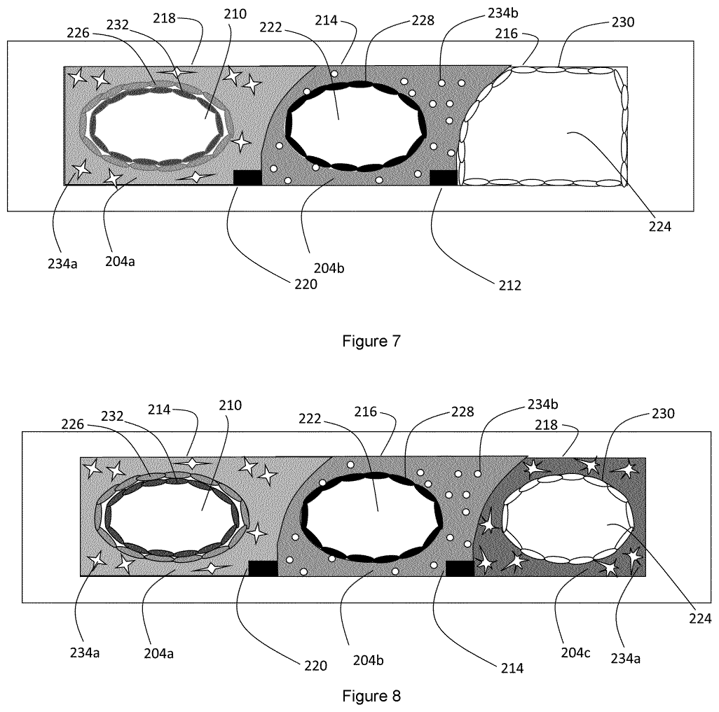

As can be seen in FIG. 7, it is also possible to introduce one or more cells into a second or third region of the microfluidic network and allow the cells to at least partially cover a second or third surface of the gel structure and the second or third region of the microfluidic network to at least partially form a tubule in the second or third region, for example wherein the one or more cells are selected from endothelial cells or epithelial cells; cells of mesenchymal origin, for example (smooth) muscle cells, pericytes, podocytes, fibroblasts, myofibroblasts.

In FIG. 7, first region 214 is denoted as being the central of three regions of the microfluidic network and has a monolayer tubule of cells 228 (for example an endothelial tubule). Second region 216 is shown as being to the right of first region 214 and is shown as being free of a gel structure. In this example, second region 216 of the microfluidic network and the second surface of the gel formed in first region 214 facing second region 216 comprises a layer of endothelial cells or epithelial cells forming a tubule. That is, cells 230 have covered the surface of region 216, and the surface of the gel structure of the first region 214, to form a tubule in second region 216.

As a result, the tubule by itself forms a lumen 224 extending through second region 216, which can be perfused with nutrient media, or with a test solution. In FIG. 7, third region 218 is provided with a gel structure and cellular system. It will be appreciated that while these Figures illustrate all tubules being substantially surrounded by cells, for example cells of mesenchymal origin, present in the gel structure, alternative models are possible in which only the first tubule and/or the second tubule are substantially surrounded by cells, for example cells of mesenchymal origin, present in the gel structure.

Generally, introducing one or more cells or cell types into the microfluidic network may comprise perfusing a liquid containing the cells through the microfluidic network from one inlet to another inlet/outlet. Perfusing the liquid may include, for example, applying a pressure difference from the inlet to the outlet, and/or adjusting the inclination of the microfluidic network. It will be appreciated that a cell-containing liquid can be perfused through any region of the microfluidic network that is provided with fluid interfaces, and through any lumen and/or cell tubule present in that region. In some examples, introducing one or more cells or cell types into the microfluidic network may comprise introducing or seeding a cell pellet into a region of the microfluidic network. In some examples, prior to seeding a cell pellet into a region, a liquid of low viscosity, such as culture media may be introduced.

In some examples, any of the first to third regions may comprise a gel structure comprising one or more types of immune cells, which may be selected from but not limited to T cells, monocytes, macrophages, neutrophils, eosinophils, mast cells, natural killer cells, dendritic cells, and B cells. The one or more types of immune cells may be present in the gel precursor solution introduced into a region of the microfluidic network, or may be provided in a lumen of the gel structure, for example a lumen lined by cells, e.g. an endothelial or lymphatic vessel and allowed or stimulated to adhere to the first surface, for example the endothelial or lymphatic vessel, allowed to extravasate from the luminal side into the ECM side. The immune cells may also be added into a region of the microfluidic network that is free from any gel structure and which is configured as a perfusion conduit for nutrient media and/or test solutions.

FIG. 8 illustrates an example apparatus comprising two capillary pressure barriers 214 and 220, which mark notional boundaries between a first region 214 of the microfluidic network, a second region 216 of the microfluidic network, and a third region 218 of the microfluidic network. All three regions have been filled with liquids comprising gel precursor solutions 204a, 204b and 204c respectively, which have subsequently been lumenized to form lumens 210, 222 and 224 and allowed to gelate.

Turning first to first region 214, it can be seen that the gel derived from gel precursor solution 204a includes cells 234a, which preferably were introduced as part of the precursor solution, but which could also be introduced after gelation, and stimulated or allowed to cross an epithelial or endothelial vessel wall, migrate into the gel structure. The cell type for cells 234a can be selected based on need. In some examples, cells 234a may include one or more cells are selected from endothelial cells or epithelial cells; cells of mesenchymal, endodermal and ectodermal origin, for example (smooth) muscle cells, pericytes, podocytes, fibroblasts, myofibroblasts, astrocytes; or one or more spheroids or organoids. In examples in which the apparatus provides a model to investigate the blood-brain-barrier, cells 234a may include human primary astrocytes dispersed through the gel matrix formed form precursor solution 204a.

Similarly, gel structures formed from precursor solutions 204b and 204c in second region 216 and third region 218 may also include one or more cells 234a and 234b dispersed through gel structures, with the cells being independently selected from endothelial cells or epithelial cells; cells of mesenchymal origin, for example (smooth) muscle cells, pericytes, podocytes, fibroblasts, myofibroblasts, astrocytes; or one or more spheroids or organoids.

Thus, the methods may include introducing cells of mesenchymal origin, for example selected from stromal cells, (smooth) muscle cells, pericytes, fibroblasts, and myofibroblasts into the microfluidic network so as to substantially surround the tubule within the lumen and/or the tubule in the second region. The cells of mesenchymal origin may be present in a gel precursor solution so as to substantially surround a tubule from the outset, or they may be introduced into the microfluidic network via a transport channel, and then migrate through a gel structure so as to substantially surround a tubule.

In some examples, the gel structures comprise epithelial cells, which during culture can proliferate and/or differentiate depending on the composition of the culture media, other cell types which may be present, and the extracellular matrix. Thus, after introduction into the microfluidic network, either using an aqueous medium, preferably a growth medium, or by using the gel (precursor), the epithelial cells are then allowed to proliferate and/or differentiate in the gel structure. Culture of the one or more types of cells or cell aggregates, for example epithelial cells, is achieved by introduction of media into the microfluidic network and continued under suitable conditions so that the cells are cultured.

In a second example, at least one type of cell or cell aggregate present in the gel or gel-precursor solution comprises epithelial cells and cells of mesenchymal origin, such as fibroblasts, smooth muscle cells, myofibroblasts, pericytes, astrocytes, oligodendrocytes and the like. Thus, after introduction into the microfluidic network by using the gel (precursor), the epithelial cells and the cells of mesenchymal origin are then allowed to interact and form a tissue that can optionally proliferate and/or differentiate.

In a further example, the at least one type of cell comprises any combination of epithelial cells and cells of mesenchymal origin, immune cells (such as T-cells, macrophages, Kuppfer cells, dendritic cells, neutrophiles, eosinophils, NK cells, B-cells, granulocytes, mast cells) and/or endothelial cells.

The methods described also include introducing one or more cells into the lumen of the gel structure, for example wherein the one or more cells comprise endothelial cells, epithelial cells or cells of mesenchymal origin, which are allowed to line the surface of the lumen and form a tubule within the lumen. In some examples, the surface of the gel facing the lumen may comprise a layer of endothelial cells or epithelial cells forming a first tubule.

As can be seen in FIG. 8, lumen 210 in first region 214 has been lined with two different cell types, to form a primary tubule of cells 226 and a secondary tubule 232 within primary tubule 226, while lumens 222 and 224 have been lined with monolayers of cells 228 and 230.

For example, cells 228 and/or 230 may be endothelial cells, and the first surface of the gel structures facing lumens 222 and 224 may be populated by endothelial cells 228 and 230 to generate endothelial tubules with open lumens. However, cells 228 and 230 may be selected to be epithelial cells, or cells of mesenchymal origin as described elsewhere, depending on the type of model being investigated.

In one example, a fluid introduced into a lumen may comprise endothelial cells. In general endothelial cells are known as the cells that line the interior surface of the entire circulatory system, from the heart to the smallest lymphatic capillaries. When in contact with blood these cells are called vascular endothelial cells and when in contact with the lymphatic system they are called lymphatic endothelial cells. In a particular embodiment the method includes the step of introducing endothelial cells into a lumen of the microfluidic network, and causing or allowing said endothelial cells to line the surface of the gel facing the lumen, i.e. causing or allowing the endothelial cells to form a tubule within lumen.

Alternatively, multilayer tubules are possible, by sequentially introducing solutions of cells into a lumen, as for lumen 210 in first region 214. For example, cells 226 may be pericytes, and lumen 210 may be first populated with pericytes 226, which are allowed or stimulated to form a monolayer or multilayer tubule in lumen 210. Cells 232 can be endothelial cells and can be introduced to generate a pericyte/endothelium-lined tube with an open lumen, where the endothelium covers the pericytes. Alternatively, multilayer tubules can be formed by introducing a solution of two different cell types (for example endothelial cells and smooth muscle cells, and allowing the different cell types to self-organise into the multi-layered tubule.

As described above, the methods may include introducing one or more immune cells into the microfluidic network. In some examples, the methods may include introducing one or more immune cells, for example T cells, monocytes, macrophages, dendritic cells, and/or B cells into a lumen and/or a gel structure so that the one or more immune cells adhere to a first surface of the gel or, when present, a tubule, or are provided in the gel structure. Once introduced into the microfluidic network, the immune cells may be stimulated or allowed to adhere to the epithelial or endothelial vessel wall of a tubule and optionally subsequently migrate across the vessel wall and into the gel structure.

Alternatively, the immune cells may adhere to an endothelium formed in the microfluidic network. The immune cells may also be stimulated or allowed to migrate through the gel structure in order to observe their behavior in another region of the microfluidic network, as will be described below in connection with the assays that may be performed using the microfluidic networks arising from the methods, and the apparatuses, of the present disclosure.

In any of the FIGS. 5 to 8 the layer 230 could be by means of example an epithelial tubule including but not limited to intestinal tubule (comprising small intestine, colon, ileum, rectum, duodenum), retinal pigment epithelium, kidney epithelium (including proximal tubule, distal tubule, loop of Henle, podocytes), skin, stomach epithelium, bile duct.

A second tubule 228 or 232 could be a blood or lymphatic vessel, that is optionally surrounded 226 by pericytes, podocytes, smooth muscle cells, cells of mesenchymal origin. Cells deposited in the interstitial space 234a and b could be means of example be cells of mesenchymal origin, such a fibroblasts, myofibroblasts, or muscle tissue, as well as resident immune cells, or immune cells that have infiltrated.

In this manner a complete functional unit a human organ can be recapitulated. For the example of an intestinal model, this model comprises all elements of relevance to the human gut, including the epithelium, submucosal layers, stromal cells, a blood vessel compartment, a lymphatic compartment and the possibility to mimic immune activation processes. The lumen of the epithelial tube could even be supplemented with mucus and microbiota.

Such a model is of importance for disease modelling (for instance inflammatory bowel disease), as well as for mimicking compound adsorption and transport. It is well known to the person skilled in the art that almost any functional unit of any organ comprises these same elements and can be modelled accordingly with help of the correct cells.

### Assays

The present invention also relates to one or more assays using an apparatus as described herein, or a lumenized gel structure produced by the methods described herein.

The assays may include a barrier function assay, a trans-epithelial electrical resistance (TEER) assay, an immune cell adhesion assay, an immune cell transmigration assay, a transporter assay, and a vasodilation or vasoconstriction assay. However, the present disclosure is not limited to the use of the present apparatus or lumenized gel structure produced by the present methods in these assays, and it will be apparent to those skilled in the art that the present invention enables any number of assays dependent on cell type, cell origin, and tests to be performed.

In some examples, any of the previously described one or more cells, or cell aggregates, introduced into the microfluidic network may comprise cell lines including immortalized cell lines or organoid lines, primary cells, induced pluripotent stem cells derived cells and may be but are not limited to clustered cells, printed cells, an organoid, tissue biopsy, tumor tissue, resected tissue material, organ explant or an embryonic body.

The one or more cells, or cell aggregates, may comprise one or more types of cells obtained from, derived from or exhibiting a phenotype associated with a particular biological tissue, for example liver, kidney, brain, breast, lung, skin, pancreas, intestine, retina or hair. The one or more cells or cell aggregates may comprise healthy or diseased tissue, and may be obtained from or derived from a patient. Cells may be of mesodermal, endodermal or ectodermal origin.

In one embodiment, the endothelial cells used to vascularise a lumen may be obtained from or derived from a patient. In one embodiment, the endothelial cells obtained from or derived from a patient may comprise blood outgrowth endothelial cells, or endothelial cells derived from pluripotent stem cells.

By using autologous endothelial cells, in combination with a biological tissue comprising one or more cells or cell aggregates from the same patient, the vascularised system is particularly suited to the field of personalised medicine and the development of clinical models and assays to determine or predict the patient's likely response to a particular agent.

For example, use of tumor tissue obtained from a patient, along with vascularisation of that tumor tissue using endothelial cells derived from the patient as described above allows for a complete analysis of the patient's likely response to a chemotherapeutic treatment. Furthermore, introduction of one or more types of the patient's own immune cells into such a system allows also for a determination to be made on a likely immune response to a given agent.

### Barrier Function Assay

Barrier function assays investigate the properties and behaviour of epithelial or endothelial cells, i.e. the cells forming an epithelium or endothelium tubule and their permeability to substances. These assays can include the addition of a dye substance such as fluorescein and observing whether or not the dye substance diffuses through the epithelium or endothelium.

The present invention enables a simple barrier function assay. For example, an apparatus may be provided with a microfluidic network as depicted in the top image of FIG. 8, in which cells 228 and 230 may be epithelial cells or endothelial cells. A diffuser dye can be introduced into lumen 222, and the system monitored for the presence of the diffuser dye in any one or more of the surrounding gel matrix, the lumen formed by the tubule of cells 230, and the free transport channel.

### Trans-Epithelial Electrical Resistance (TEER) Assay

TEER assays are specific examples of barrier function assays, and investigate barrier permeability by monitoring the electrical resistance across an epithelial cell layer. TEER assays performed on cell layers in microfluidic networks are described in WO 2019/166644, for example, the contents of which are incorporated herein by reference. It can be seen from FIG. 2 of WO 2019/166644 that a 3-lane microfluidic network, such as those described herein, can be provided with up to six electrode pairs for measuring electrical activity in a TEER barrier function assay.

### Immune Cell Adhesion Assay

As has been described already, the methods of the invention can include introducing immune cells into the microfluidic network. The immune cells may be as described above, and may be introduced into the microfluidic network at any location of the network as described above.

Immune cell adhesion, for example, T-cell adhesion to epithelial or endothelial tissue is a critical step in the inflammatory response, and so it is desirable to have systems, for example apparatuses comprising microfluidic networks, that enable investigations into immune cell adhesion to these tissues.

The present invention therefore also relates to use of the apparatuses and microfluidic networks described herein in assays to investigate immune cell adhesion to biological structures.

### Immune Cell Transmigration Assay

As has been described already, the methods of the invention can include introducing immune cells into the microfluidic network. The immune cells may be as described above, and may be introduced into the microfluidic network at any location of the network as described above.

The present invention enables investigations into the kinetics and regulatory mechanisms of immune cell migration through the epithelium and endothelium.

There is considerable interest in developing reliable immunotherapies against major diseases, and so immune cell transmigration assays, in particular assays looking at regulatory T cell migration can help to understand the underlying functional roles of Tregs in autoimmune diseases, including multiple sclerosis; type 1 diabetes; rheumatoid arthritis and cancers such as lung cancer, colorectal cancer, nasopharyngeal carcinoma and breast cancer. For example, to prevent allograft rejection, Tregs must migrate to both grafts and lymph nodes. Furthermore, the migration and accumulation of functionally suppressive Tregs at tumor sites is associated with the advancement of cancer.

The present invention therefore also relates to use of the apparatuses and microfluidic networks described herein in assays to investigate immune cell transmigration.

### Transporter Assay

As has been described already, the present disclosure relates to the formation of monolayer biological structures, and more complex multi-layered biological structures with the lumen of the lumened gel structures of the microfluidic network. These biological structures can recreate the in vivo environment of, for example, the blood-brain barrier, the intestine, as well as other organs such as the kidney or liver.

Understanding how potential therapeutic drugs are transported across such biological structures is of high clinical importance, as a lack of permeability across any given biological membrane or barrier will result in a reduced efficacy of the drug candidate. Formation of biologically relevant tissues in the lumened gel structure of the microfluidic networks described enables pharmacokinetic investigations into which transporter proteins are involved in any drug transport. For instance, a compound could be administered to the apical side of a tubule, for instance an intestinal tubule, and its concentration could be measured in a blood vessel that is present in the lumenized ECM. The compound could be quantified by means of fluorescent imaging, mass spectrometry, ELISA.

### Vasodilation or Vasoconstriction Assay.

The described apparatuses and microfluidic networks allow for the formation of lumened biological tissues based on epithelial or endothelial cells, or muscle cells, fibroblasts, cardiomyocytes, for example, to result in an endothelial blood vessel, while epithelial cells can form an intestinal type lumen or a kidney tubule type lumen, and cardiomyocytes can form an atrial or ventricular type lumen.

Microfluidic networks bearing such tissues are therefore particularly well suited to the study of processes such as vasodilation/vasoconstriction, gut peristaltic motion, kidney tubule compression, vascular compression, or cardiomyocyte actuation. The (very thin) ECM supporting the tubule can easily be deformed by a constricting or dilating tubule or other moving tissue, which enables more sensitive assays of tissue motility than constructs where cells are directly attached to more rigid surfaces.

The present invention therefore also relates to use of the apparatuses and microfluidic networks described herein in vasodilation or vasoconstriction assays, as well as assays investigating gut peristaltic motion, kidney tubule compression, vascular compression, and cardiomyocyte actuation, to investigate the effect of a stimulant or suppressant on a biological system.

### EXAMPLES

The present invention will now be described by way of example only with reference to the following examples.

### Example 1

### Seeding Cells in a Lumen and Cells Against Gel

### Materials:

EGM-2™ Medium:
EBM-2 basal medium (Lonza, Cat. No. CC-3156)
EGM-2 SingleQuots supplements (Lonza, Cat. No. CC-4176)
1% Penicillin/Streptomycin (Sigma, Cat. No. P4333)

### EMEM:

Eagle's Minimum Essential Medium (EMEM) (ATCC, Cat. No. 30-2003)
10% Foetal Bovine Serum (Gibco, Cat. No. 16140-071)
1% Non-Essential Amino Acids (LifeTech, Cat. No. 11140050)
1% Penicillin/Streptomycin (Sigma, Cat. No. P4333)
HBSS (Hanks' Balanced Salt Solution; Sigma Aldrich, Cat. No. H6648)
PBS (Phosphate buffered saline; Gibco, Cat. No. 700130656)

For this example, a 3-lane OrganoPlate® (MIMETAS) with 400 μm wide lanes was used, which comprises a microfluidic network of 3 lanes and two capillary pressure barriers.

The central lane was filled at the inlet with a neutralised bovine type I atelocollagen solution (PureCol® EZ Gel 5 mg/ml from Advanced BioMatrix); the solution was pinned at the capillary pressure barrier leaving a free-standing meniscus at the air-liquid interface in the upper and lower lane.

A 30 μL solution of HBSS was introduced into the inlet of the centre lane, and a 1 μL solution of 5% FBS in PBS was introduced at the hole of the outlet of the centre lane forming a dome shape. These solutions are of lower viscosity than the PureCol® EZ Gel This resulted in a pressure from the outlet towards the inlet due to the surface tension of the droplet and the resultant Laplace pressure, initiating lumenization. After 1 minute 50 μL of HBSS was added to the outlet and the OrganoPlate was placed in a humidity-controlled environment at 37° C. and 5% CO₂ for 1 hour.

Representative measurements on the rheology of ECMs published by Fleming et al (https://doi.org/10.1063/1.5067382). Viscosities of commonly used cell culture media are e.g. published by Poon (https://doi.org/10.1101/2020.08.25.266221).

For cell seeding, 1 μL of a Primary Human Umbilical Vein Endothelial Cells (HUVEC) cell pellet containing 10,000 cell/μL was seeded in the outlet of the middle lane.

Cells were left to attach for between 2-6 hours. Following this, EGM-2 cell culture media was added to the middle lane so that the volume at the inlet and outlet was 50 μL, and the OrganoPlate® placed on a rocking platform with a 7°, 8 minutes interval so that cell culture media could be passively perfused through the lumen.

After 2 days Caco-2 cells were added to the top lane inlet by the same means, seeding a Caco-2 cell pellet containing 6,000 cells/μL into the top lane outlet. Cells were left to attach for between 2-6 hours. After attaching, complete EMEM media was added to the top lane so that both inlet and outlet contained 50 μL of complete EMEM media. The OrganoPlate® was placed on rocking platform with a 7°, 8 minutes interval so that cell culture media could be passively perfused through the lumen.

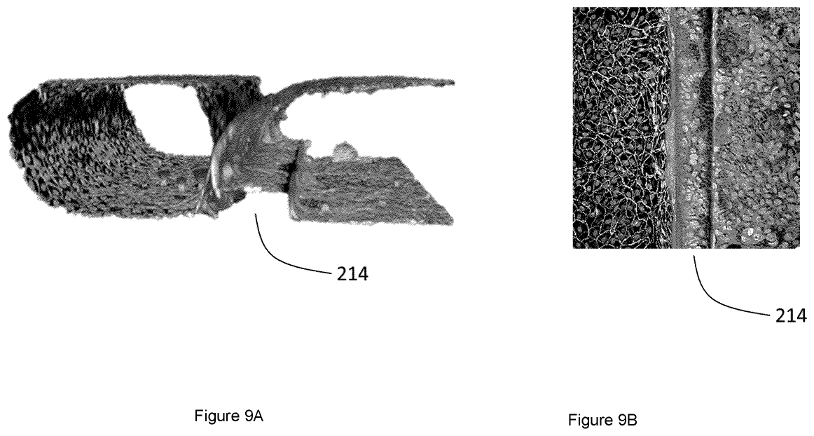

Within 1-3 days both types of cells form tubules within the OrganoPlate®, separated by <200 μm ECM. Cell culture was maintained for 10 days before fixation with 3.7% Formalin solution. Caco-2 cells were stained for epithelial cell adhesion molecule (EPCAM) and HUVEC for VE-Cadherin and the resultant confocal 3D reconstruction can be seen in FIGS. 9A and 9B. FIG. 9A shows the view along the tubules, while FIG. 9B shows the view from above. In both Figures, the left hand tubule is formed from Caco-2 cells, and the right hand tubule from HUVEC cells. The location of the capillary pressure barrier 214 and the geometry it causes the tubules, in particular the HUVEC tubule, to adopt can clearly be seen.

It can be also seen from these figures that the Caco-2 cells and HUVEC cells are in close contact, facilitating cell-to-cell communications in a tissue culture model more closely resembling the in vivo situation. This was measured to be between 15-150 μm, depending on the length of storage time.

In another example Caco-2 cells were seeded into the bottom lane, in the same manner as described above, to form a construct consisting of 3 tubules, see FIG. 10). This illustrates the utility of the third lane in also supporting a tubule.

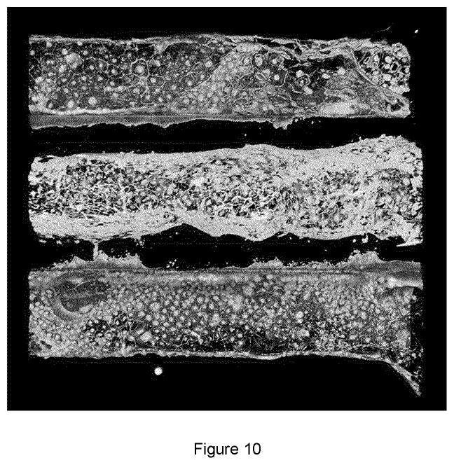

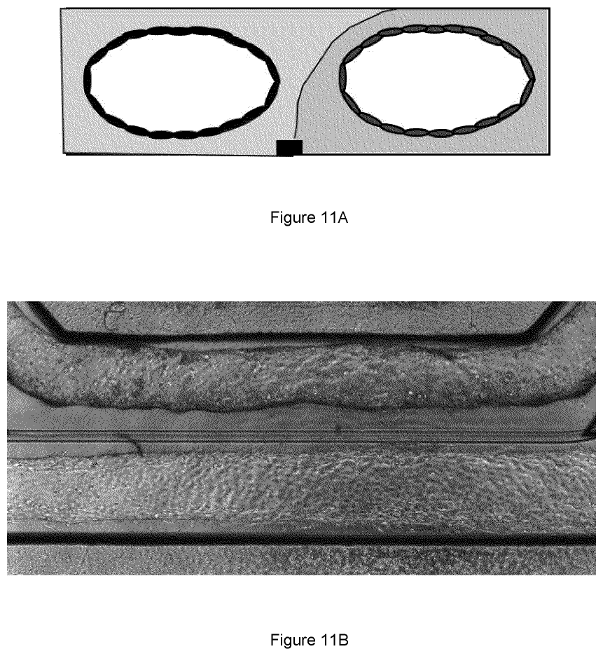

Two further examples show how two and even three channels can be lumenized subsequently. FIG. 11A shows a schematic of two Caco-2 tubules in lumenized ECM in a 2-lane OrganoPlate, with the experimentally obtained phase contrast microscopy image in plan view shown in FIG. 11B. Following the procedure set out above a Purecol EZ solution was introduced in the top channel and a lumen was formed using viscous finger patterning. Next Purecol EZ solution was introduced in the bottom channel and a lumen was formed. Caco-2 cells were seeded in the lumen of the top channel and HUVEC cells were seeded in the lumen of the bottom channel. This way, a construct of two lumened ECMs both comprising a tubule was formed.

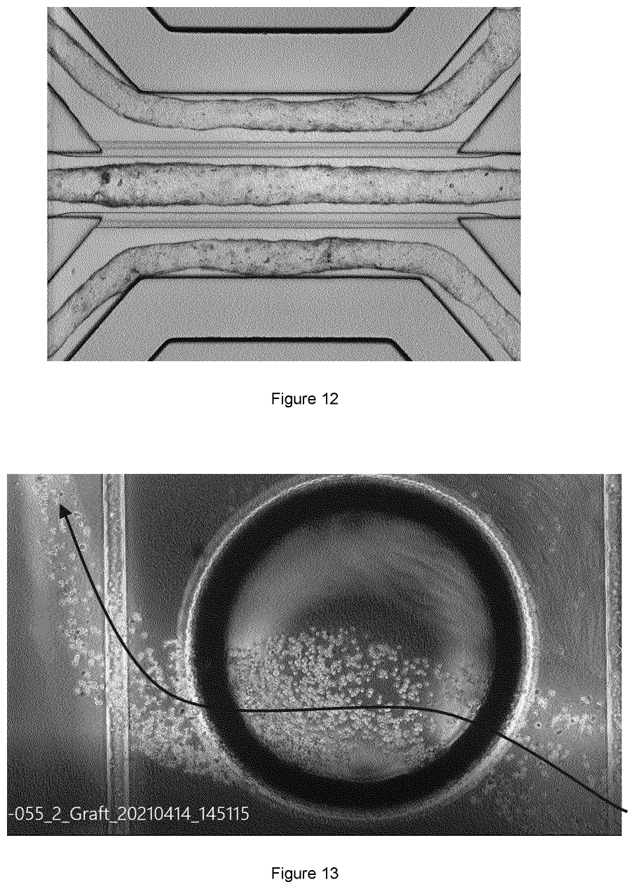

In a similar manner FIG. 12 shows a construct of three lumened ECMs, all three comprising a tubule consisting of Caco-2 cells.

### Example 2

### Lumenised Blood-Brain-Barrier Model

### Endothelial Medium:

Endothelial cell medium (Cell biologics, Ca. No. H1168)
Endothelial cell medium supplement kit (Cell biologics, Ca. No. H1168)

### NSC Differentiation Medium:

In-house proprietary formulation, comprising
Neurobasal medium (Gibco, Cat. No. 21103049)

For this example, a 3-lane OrganoPlate® (MIMETAS) with 400 μm wide lanes was used, which comprises a microfluidic network of 3 lanes and two capillary pressure barriers.

The top lane was filled at the inlet with a neutralised bovine type I atelocollagen solution PureCol® EZ Gel 5 mg/ml (Advanced BioMatrix); the solution was pinned at the capillary pressure barrier leaving a free-standing meniscus at the air-liquid interface in the upper and lower lane.

A 30 μL solution of HBSS was introduced into the inlet of the top lane, and a 1 μL solution of 5% FBS in PBS was introduced at the hole of the outlet of the top lane forming a dome shape as in Example 1. This resulted in a pressure from the outlet towards the inlet due to the surface tension of the droplet and the resultant Laplace pressure. After 1 minute 50 μL of HBSS was added to the outlet and the OrganoPlate was placed in a humidity-controlled environment at 37° C. and 5% CO₂ for 1 hour.

For cell seeding of astrocytes in gel, 2 μL of an astrocytes cell suspension in Collagen I 4 mg/mL (Cultrex) containing 5,000 cells/μL was seeded in the outlet of the middle lane.

Cells were left to attach in a humidity-controlled environment at 37° C. and 5% CO₂, for between 2-6 hours.

Then, 2 μL of a neurons cell pellet containing 15,000 cell/μL was seeded in the outlet of the bottom lane.

Cells were left to attach for between 2-6 hours. Following this, NSC differentiation media (Gibco) was added to the bottom lane so that the volume at the inlet and outlet was 50 μL, and the OrganoPlate® placed on rocking platform with a 7°, 8 minutes interval so that cell culture media could be passively perfused through the lumen.

Within 1-7 days the astrocytes formed a cellular network in the gel of the central lane and the neurons in the bottom lane. After 7 days Human Brain Microvascular Endothelial Cells (HBMEC) were seeded in the top lane by the same means, by seeding 1 μL of a HBMEC cell pellet containing 10,000 cells/μL into the top lane outlet. Cells were left to attach for between 2-6 hours.

Once cells attached, cell culture media (Cell Biologics) was added to the top lane so that both inlet and outlet contained 50 μL of cell culture media (Cell Biologics). The OrganoPlate® was placed on a rocking platform with a 7°, 8 minutes interval so that cell culture media could be passively perfused through the lumen. Refreshment of media in the top and bottom lane was performed every 2-3 days.

Within 1-3 days the HBMECs formed tubules within the OrganoPlate® with direct contact to the astrocytes in the gel. Cell culture was maintained for 14 days and barrier integrity assays were performed on the HBMECs using sodium fluorescein (dilution 1:100) and 4.4 kDa TRITC (dilution 1:50).

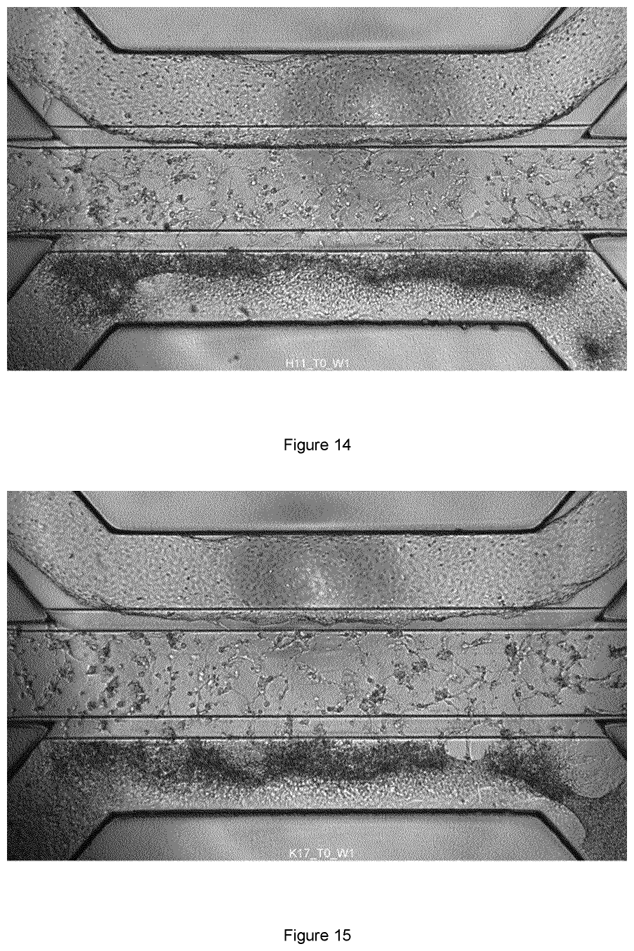

FIG. 14 (astrocytes in Collagen I) and FIG. 15 (astrocytes in PureCol®) are high resolution images showing the results of these experiments. It can be seen from both Figures that the HBMECs and astrocytes are in close contact, facilitating cell-to-cell communications in a tissue culture model more closely resembling the in vivo situation.

### Example 3

### Tube in Tube Endothelial Cell/Pericyte Co-Culture

### MV2 Medium:

MV2 (Promocell, Cat. No. C22022)
Supplement mix (Promocell, Cat. No. C39226)
1% Penicillin/Streptomycin (Sigma, Cat. No. P4333)
   Pericyte medium:
Pericyte growth medium (Promocell, Cat. No. C-28041)

For this example, a 3-lane OrganoPlate® (MIMETAS) with 400 μm wide lanes was used, which comprises a microfluidic network of 3 lanes and two capillary pressure barriers.

The top lane was filled at the inlet with a neutralised bovine type I atelocollagen solution PureCol® EZ Gel 5 mg/ml (Advanced BioMatrix); the solution was pinned at the capillary pressure barrier leaving a free-standing meniscus at the air-liquid interface in the upper and lower lane.

A 30 μL solution of HBSS was introduced into the inlet of the top lane, and a 1 μL solution of 5% FBS in PBS was introduced at the hole of the outlet of the top lane forming a dome shape as in Example 1. This resulted in a pressure from the outlet towards the inlet due to the surface tension of the droplet and the resultant Laplace pressure. After 1 minute 50 μL of HBSS was added to the outlet and the OrganoPlate was placed in a humidity-controlled environment at 37° C. and 5% CO₂ for 1 hour.

For cell seeding of pericytes and Human Umbilical Vein Endothelial Cells (HUVEC), 1 μL of a cell pellet containing 15,000 cell/μL HUVECs and 5,000 cells/μL of placenta pericytes was seeded in the outlet of the top lane. The cell suspension was driven into the microfluidic network by means of Laplace pressure due to the surface tension of the cell pellet.

Cells were left to attach for between 2-6 hours. Following this, a 1:1 mix of MV2 medium and pericyte growth medium was added to the top lane so that the volume at the inlet and outlet was 50 μL, and the OrganoPlate® was placed on a rocking platform with a 7°, 8 minutes interval so that cell culture media could be passively perfused through the lumen.

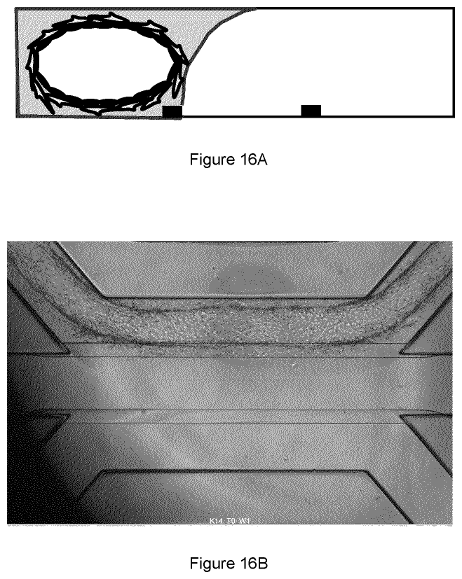

Within 1-3 days the cells organized themselves in the co-culture. The HUVECs formed tubules within the OrganoPlate® and the placenta pericytes migrated into the ECM and surrounded the cell tubule. The middle and bottom channel are filled with medium. FIG. 16A shows a schematic cross-sectional view and FIG. 16B shows an experimentally obtained confocal microscopy cross-sectional view of the tube-in-tube HUVEC/pericyte co-culture.

### Example 4

Triple Co-Culture with Stromal Cells in Gel

### MV2 Medium:

MV2 (Promocell, Cat. No. C22022)
Supplement mix (Promocell, Cat. No. C39226)
1% Penicillin/Streptomycin (Sigma, Cat. No. P4333)

### EMEM:

Eagle's Minimum Essential Medium (EMEM) (ATCC, Cat. No. 30-2003)
10% Foetal Bovine Serum (Gibco, Cat. No. 16140-071)
35 1% Non-Essential Amino Acids (LifeTech, Cat. No. 11140050)
1% Penicillin/Streptomycin (Sigma, Cat. No. P4333)

For this example, a 3-lane OrganoPlate® (MIMETAS) with 400 μm wide lanes was used, which comprises a microfluidic network of 3 lanes and two capillary pressure barriers.

The middle lane was filled at the inlet with a neutralised bovine type I atelocollagen solution PureCol® EZ Gel 5 mg/ml (Advanced BioMatrix) containing intestinal fibroblasts at a concentration of 5,000 cells/μL. The solution was pinned at the capillary pressure barrier leaving a free-standing meniscus at the air-liquid interface in the upper and lower lane. A 30 μL solution of HBSS was introduced into the inlet of the middle lane, and a 1 μL solution of 5% FBS in PBS was introduced at the hole of the outlet of the middle lane forming a dome shape as in Example 1. This resulted in a pressure from the outlet towards the inlet due to the surface tension of the droplet and the resultant Laplace pressure. After 1 minute 50 μL of HBSS was added to the outlet and the OrganoPlate was placed in a humidity-controlled environment at 37° C. and 5% CO₂ for 1 hour.

For cell seeding, 1 μL of a Primary Human Umbilical Vein Endothelial Cells (HUVEC) cell pellet containing 10,000 cell/μL was seeded in the outlet of the middle lane. Cells were left to attach for between 2-6 hours. Following this, MV2 cell culture media was added to the middle lane so that the volume at the inlet and outlet was 50 μL, and the OrganoPlate® placed on a rocking platform with a 7°, 8 minutes interval so that cell culture media could be passively perfused through the lumen. After 2 days Caco-2 cells were added to the top lane inlet by the same means, seeding a Caco-2 cell pellet containing 6,000 cells/μL into the top lane outlet. Cells were left to attach for between 2-6 hours. After attaching, complete EMEM media was added to the top lane so that both inlet and outlet contained 50 μL of complete EMEM media. The OrganoPlate® was placed on rocking platform with a 7°, 8 minutes interval so that cell culture media could be passively perfused through the lumen.

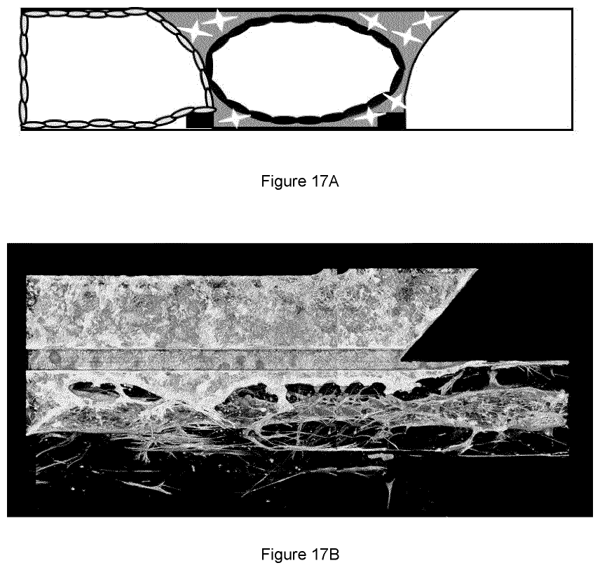

Within 1-3 days both types of cells form tubules within the OrganoPlate®, separated by <200 μm ECM. The gel contained the fibroblasts which were surrounding the HUVEC tubule and were between the HUVEC and Caco-2 tubule. FIG. 17A shows a schematic cross-sectional view of the triple co-culture.

Cell culture was maintained for 7 days before fixation with 3.7% Formalin solution. Caco-2 cells were stained for epithelial cell adhesion molecule (EPCAM), HUVEC for VE-Cadherin and all three cell types including fibroblasts were stained for actin. The resultant confocal 3D reconstruction is shown in FIG. 17B.

### Example 5

### Visualising Contractile Lumen

### MV2 Medium:

MV2 (Promocell, Cat. No. C22022)
Supplement mix (Promocell, Cat. No. C39226)
1% Penicillin/Streptomycin (Sigma, Cat. No. P4333)

For this example, a 3-lane OrganoPlate® (MIMETAS) with 400 μm wide lanes was used, which comprises a microfluidic network of 3 lanes and two capillary pressure barriers. The network was filled with 50 μL HBSS in the observation window (the point where the 3 lanes converge) to provide additional humidity to the plate.

The central lane was filled at the inlet with a neutralised bovine type I atelocollagen solution PureCol® EZ Gel 5 mg/ml (Advanced BioMatrix); the solution was pinned at the capillary pressure barrier leaving a free-standing meniscus at the air-liquid interface in the upper and lower lane.

A 30 μL solution of HBSS was introduced into the inlet of the centre lane, and a 1 μL solution of 5% FBS in PBS was introduced at the hole of the outlet of the centre lane forming a dome shape as in Example 1. This resulted in a pressure from the outlet towards the inlet due to the surface tension of the droplet and the resultant Laplace pressure. After 1 minute 50 μL of HBSS was added to the outlet and the OrganoPlate was placed in a humidity-controlled environment at 37° C. and 5% CO₂ for 1 hour.

For cell seeding, 1 μL of a cell suspension containing 10,000 cell/μL Primary Human Coronary Artery Endothelial Cells (HCAEC) and 2,500 cell/μL Primary Human Coronary Artery Smooth Muscle Cells (HCASMC) was seeded in the outlet of the middle lane. The cell suspension was driven into the microfluidic network by means of Laplace pressure due to the surface tension of the cell pellet.

Cells were left to attach for between 2-6 hours. Following this, MV-2 cell culture media was added to the middle lane so that the volume at the inlet and outlet was 50 μL, and the OrganoPlate® was placed on rocking platform with a 7°, 8 minutes interval so that cell culture media could be passively perfused through the lumen. Within 1-3 days cells form tubules within the lumen created within the OrganoPlate®.

Due to the ECM within the lumen being <200 μm it is possible to visualise the contraction of the tubule upon exposure of a stimulus. For this, cells were firstly exposed at day 3 to Norepinephrine at concentrations of 1 μM, 10 μM and 100 μM, and then exposed to Sildenafil at day 6 in concentrations of 5 μM, 50 μM and 500 μM including medium only and vehicle controls. Images were taken using a phase contrast ImageExpress Nano microscope (Molecular devices) at 1-minute intervals for 30 minutes.

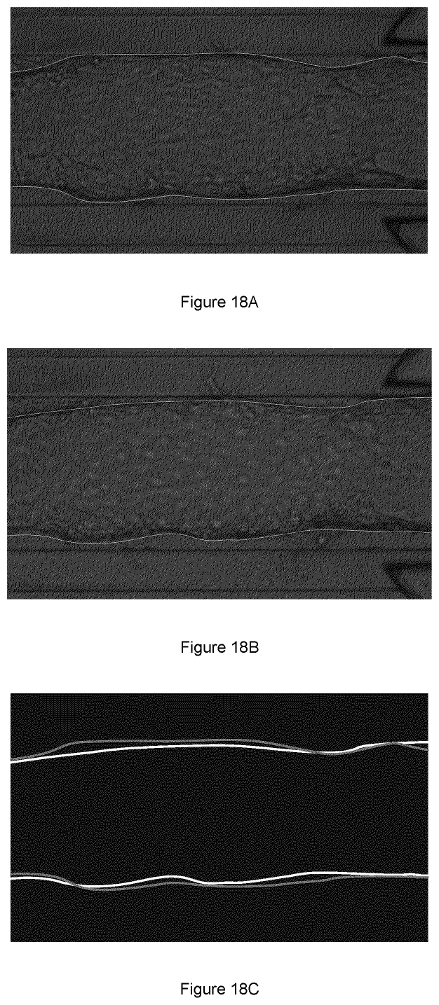

FIGS. 14A to 14C demonstrate the contraction of the HCAEC and HCASMC co-culture following the exposure to Sildenafil. FIG. 18A was taken before exposure; FIG. 18B as taken at 30 minutes post-exposure; and FIG. 18C shows a composite outline demonstrating that the diameter of the lumen changes upon exposure to a stimulus. Visualisation of this was made possible due to the small forces required as a result of the thin ECM produced during lumenisation.

### Example 6

T-Cell Migration from an Epithelial Tubule into Tumor Cells
MCDB131 medium:
MCDB131 (Thermo Fischer, Cat. No. 10372019)
10 ng/ml hEGF (Sigma, Cat. No. E9644)
1 μg/ml Hydrocortisone (Sigma, Cat. No. H0135)
10 mM L-glutamine (Sigma, Cat. No. G7513)
10% Foetal Bovine Serum (ATCC, Cat. No. 30-2020)
1% Penicillin/Streptomycin (Sigma, Cat. No. P4333)
   AIM-V medium:
AIM-V (Thermo Fischer, Cat. No. 12055091)

For this example, a 3-lane OrganoPlate® (MIMETAS) with 400 μm wide lanes was used, which comprises a microfluidic network of 3 lanes and two capillary pressure barriers.

The central lane was filled at the inlet with a neutralised bovine type I atelocollagen solution PureCol® EZ Gel 5 mg/mL (Advanced BioMatrix); the solution was pinned at the capillary pressure barrier leaving a free-standing meniscus at the air-liquid interface in the upper and lower lane.

A 30 μL solution of HBSS was introduced into the inlet of the centre lane, a 1 μL of 5% FBS in PBS was introduced at the hole of the outlet of the centre lane forming a dome shape as in Example 1. This resulted in a pressure from the outlet towards the inlet due to the surface tension of the droplet and the resultant Laplace pressure. After 1 minute 50 μL of HBSS was added to outlet and the OrganoPlate was placed in a humidity-controlled environment at 37° C. and 5% CO₂ for 1 hour.

For cell seeding, 1 μL of a Primary Human Mammary Epithelial Cell (HMEC) pellet containing 10,000 cell/μL was seeded in the outlet of the top. Cells were left to attach for between 2-6 hours. Following this, MCDB131 cell culture media was added to the top lane so that the volume at the inlet and outlet was 50 μL, and the OrganoPlate® was placed on rocking platform with a 7°, 8 minutes interval so that cell culture media could be passively perfused through the lumen.

After 5 days, A375 tumor cells were added to the middle inlet by the same means. To this end, 2.5 μL of a A375 tumor cell pellet containing 10,000 cells/μL was seeded into the middle lane outlet. Cells were left to for between 2-6 hours. Once cells attached, AIM-V cell culture media (Thermo Fischer) was added to the middle lane so that both inlet and outlet contained 50 μL of AIM-V cell culture media (Thermo Fischer). The OrganoPlate® was placed on rocking platform with a 7°, 8 minutes interval so that cell culture media could be passively perfused through the lumen. Within 1-3 days the epithelial cells form tubules within the OrganoPlate® and were separated from the tumor cells by a <200 μm ECM.

Due to the ECM separating the two cell types being <200 μm it is possible to perform a T-cell migration assay to investigate the number of T-cells migrating from the epithelial tubule to the tumor cell compartment. For this, on day 6 T-cells isolated from a buffy coat were fluorescently labelled by resuspending the T-cell pellet in 2.5 μM CellTracker CMRA (Invitrogen). The labelled T-cells were added to the top inlet, i.e. to the epithelial tubule in the top lane. First, 50 μL of a T-cell suspension in AIM-V cell culture medium containing 400,000 cells/mL was added in the top lane inlet. Then, 50 μL of AIM-V cell culture media (Thermo Fischer) was added to the top lane outlet. Cells were left to enter the epithelial tubule.

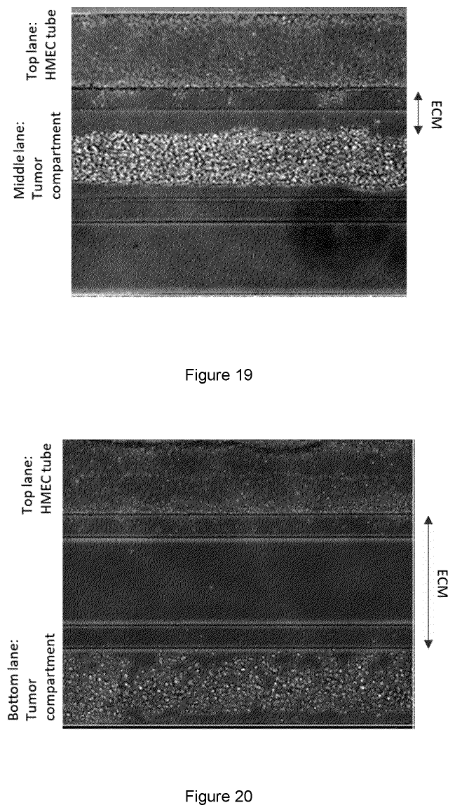

Images were taken using a phase contrast ImageExpress Nano microscope (Molecular devices) at 24 h, 48 h and 72 h after T-cell seeding. It can be seen from FIG. 19 that the epithelial cell compartment and the tumor cell compartment are in close proximity resembling the in vivo situation, with T-cells migrating through the ECM into the tumor cell compartment.

As a comparison, see FIG. 20, the same experiment was performed on a regular 3-lane OrganoPlate®, but with the top lane containing an epithelial tubule (as in FIG. 19) being separated from the tumor cells (present this time in the bottom lane) by an approx. 400 μm ECM in the middle lane.

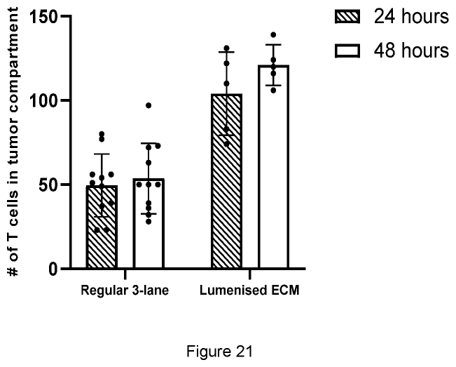

FIG. 21 shows the graphs of the quantification of T-cell migration in the systems of FIGS. 15 and 16 respectively. As can be seen in FIG. 21, twice the amount of T-cells was observed in the tumor compartment formed using the inventive methods (FIG. 21 “Lumenised ECM”) after 24 hours and 48 hours compared to the experiment where the epithelial cells and tumor cells were separated by a 400 μm wide ECM (FIG. 21 “Regular 3-lane”).

The present invention thus enables the co-culture of multiple types of primary cell types, for example human brain-derived vascular cells (e.g., endothelial cells, pericytes and astrocytes), and tumor models, in a microfluidic network to create a system in which their normal 3D spatial relationships—in particular a thin interstitial layer of only tens of microns, for example 50 μm or less—are maintained. This is all achieved without the use of a structural membrane, so that the behaviour of these cells can be investigated in vitro while faithfully mimicking the in vivo environment.

The above examples are intended for the purpose of teaching a person of ordinary skill in the art how to practice the present invention, and it is not intended to detail all those modifications and variations which will become apparent upon reading the description. It is intended, however, that all such modifications and variations be included within the scope of the present invention, which is defined in and by the following claims.

1. A method for creating a lumenized gel structure, comprising:
introducing a first liquid comprising a gel precursor solution into a microfluidic network, the microfluidic network comprising a capillary pressure barrier at a position generally defining a boundary between first and second regions of the microfluidic network;
allowing the first liquid to enter the first region of the microfluidic network and align itself along the capillary pressure barrier, thereby forming a liquid-air meniscus of the first liquid at the boundary between the first and second regions of the microfluidic network;
forming a lumen through the first liquid by contacting the first liquid with a second liquid, wherein the second liquid has a viscosity which is lower than the viscosity of the first liquid; and
allowing or causing the first liquid to gelate to form a gel structure comprising a lumen therethrough.

2. The method of claim 1, wherein the gel structure comprises a first surface facing the lumen and a second surface facing the second region of the microfluidic network.

3. The method of claim 2, wherein the gel structure has a thickness between the first surface and the second surface of 200 μm or less, preferably less than 100 μm.

4. The method of claim 2 or claim 3, wherein:
a second capillary pressure barrier is provided at a position generally defining a boundary between the first region and a third region of the microfluidic network; and
the first liquid aligns itself along the second capillary pressure barrier, thereby forming a third surface of the gel structure facing the third region of the microfluidic network.

5. The method of any one of the preceding claims, wherein the gel comprises an extracellular matrix, optionally selected from one or more of collagen I, collagen IV, fibrin, fibrinogen, laminin, Matrigel®, hyaluronic acid, and a synthetic hydrogel.

6. The method of any one of the preceding claims, wherein contacting the gel precursor solution with the second liquid comprises:
forming a meniscus of the second liquid that is convex in shape and has a first principal radius of curvature.

7. The method of any one of the preceding claims, wherein forming the lumen comprises:
contacting the gel precursor solution with the second liquid at a first location in the microfluidic network; and
contacting the gel precursor solution with a third liquid at a second location spaced from the first location, wherein the third liquid has a viscosity which is lower than the viscosity of the first liquid.

8. The method of claim 7 when dependent on claim 6, wherein contacting the gel precursor solution with the third liquid comprises:
forming a meniscus of the third liquid that is concave in shape; or that is convex in shape with a second principal radius of curvature that is smaller than the first principal radius of curvature.

9. The method of any one of the preceding claims, wherein forming the lumen comprises passively pumping the second liquid through the first liquid using surface tension or gravity.

10. The method of any one of the preceding claims, further comprising:
introducing a second gel precursor solution into the second region of the microfluidic network and allowing the second gel precursor solution to contact the gel structure along the length of the capillary pressure barrier;
forming a lumen through the second gel precursor solution by contacting the second gel precursor solution with a liquid having a viscosity which is lower than the viscosity of the second gel precursor solution; and
allowing or causing the second gel precursor solution to gelate, to form a second gel structure comprising a lumen therethrough and contacting the first gel structure.

11. The method of any of the preceding claims, wherein the second liquid comprises a gel precursor solution.

12. The method according to claim 11, further comprising:
forming a lumen through the second liquid by contacting the second liquid with a liquid having a viscosity which is lower than the viscosity of the second liquid; and
allowing or causing the second liquid to gelate within the first gel structure to form a laminar gel structure comprising a lumen therethrough.

13. The method of any one of the preceding claims, wherein the gel precursor solution comprises one or more cells of mesenchymal origin, for example selected from stromal cells, muscle cells, pericytes, fibroblasts, and myofibroblasts.

14. The method of any one of the preceding claims, further comprising:
introducing one or more cells into the lumen of the gel structure, for example wherein the one or more cells comprise endothelial cells or epithelial cells.

15. The method of claim 14, wherein the one or more cells comprise endothelial cells or epithelial cells and the method further comprises:
allowing the one or more cells to line the surface of the lumen and form a tubule within the lumen.

16. The method of any one of claims 2 to 15, further comprising:
introducing one or more cells into the second region of the microfluidic network and allowing the cells to at least partially cover the second surface of the gel structure and the second region of the microfluidic network to at least partially form a tubule in the second region, for example wherein the one or more cells are selected from endothelial cells or epithelial cells; cells of mesenchymal origin, for example (smooth) muscle cells, pericytes, podocytes, fibroblasts, myofibroblasts, astrocytes; or one or more spheroids or organoids.

17. The method of any one of claims 14 to 16, further comprising:
allowing or stimulating the one or more cells to remodel the gel structure by reducing the thickness of the gel structure between the first surface and the second surface and/or by secreting one or more ECM components.

18. The method of any one of claims 15 to 17, further comprising:
introducing cells of mesenchymal origin, for example selected from stromal cells, muscle cells, pericytes, fibroblasts, and myofibroblasts into the microfluidic network so as to substantially surround the tubule within the lumen and/or the tubule in the second region.

19. The method of any one of the preceding claims, further comprising:
introducing one or more immune cells, for example T cells, monocytes, macrophages, dendritic cells, and/or B cells into the lumen and/or the gel so that the one or more immune cells adhere to the first surface of the gel or, when present, the tubule, or are provided in the gel structure.

20. The method of claim 19, further comprising:
stimulating or allowing the one or more immune cells to cross the epithelial or endothelial vessel wall of the tubule, and optionally to migrate through the gel structure.

21. The method of any one of the preceding claims, wherein the capillary pressure barrier is provided on an internal surface of the microfluidic network and comprises a ridge, groove, or line of material that increases the contact angle of water with the internal surface of the microfluidic network.

22. The method of any one of the preceding claims, wherein the microfluidic network comprises at least two inlets and wherein the at least two inlets are connected to the first region.

23. The method of any one of the preceding claims, wherein the microfluidic network comprises at least three inlets and wherein two inlets of the at least three inlets are connected to the first region and a third inlet of the at least three inlets is connected to the second region,
preferably wherein the microfluidic network comprises four inlets and wherein two inlets of the at least four inlets are connected to the first region and wherein two inlets of the at least four inlets are connected to the second region.

24. An apparatus, comprising:
a microfluidic network, the microfluidic network comprising
at least two inlets;
a capillary pressure barrier at a position defining a boundary between first and second regions of the microfluidic network; and
a gel provided in the first region extending between two inlets of the at least two inlets and confined to the first region by the capillary pressure barrier;
wherein the gel comprises a lumen extending therethrough between the two inlets of the at least two inlets; the gel having a first surface facing the lumen, a second surface facing the second region of the microfluidic network, and a thickness of the gel between the first surface and the second surface is 200 μm or less.

25. The apparatus of claim 24, wherein the apparatus is configured to allow formation of or comprises a 3D constituted tissue having a thin interstitial space.

26. The apparatus of claim 24 or claim 25, wherein the second surface of the gel extends the length of the capillary pressure barrier.

27. The apparatus of any one of claims 24 to 26, wherein a thickness of the gel between the first surface and the second surface is 100 μm or less, for example 50 μm or less.

28. The apparatus of any one of claims 24 to 27, wherein the lumen is substantially cylindrical, for example wherein the lumen comprises a substantially circular cross-section.

29. The apparatus of any one of claims to 24 to 28, wherein the microfluidic network comprises an aperture and the gel structure forms a surface facing and/or substantially sealing the aperture.

30. The apparatus of claim 29, wherein the lumen does not extend to the aperture.

31. The apparatus of any one of claims 24 to 30 wherein the capillary pressure barrier is provided on an internal surface of the microfluidic network and comprises a ridge, groove, or line of material that increases the contact angle of water with the internal surface of the microfluidic network.

32. The apparatus of any one of claims 24 to 31, wherein the capillary pressure barrier is patterned on an internal surface of the microfluidic network to mimic a biological structure, for example wherein the capillary pressure barrier comprises a sinusoidal shape to mimic crypt villi structures.

33. The apparatus of any one of claims 24 to 32, wherein the surface of the gel facing the lumen comprises a layer of endothelial cells or epithelial cells forming a first tubule.

34. The apparatus of any one of claims 24 to 44, wherein the second region of the microfluidic network and the second surface of the gel facing the second region comprises a layer of endothelial cells or epithelial cells forming a second tubule.

35. The apparatus according to claim 33 or claim 34, wherein the first tubule and/or the second tubule are substantially surrounded by cells of mesenchymal origin, for example wherein the cells of mesenchymal origin are present in the gel structure.

36. The apparatus of claim 34 or claim 35, wherein a distance from the first tubule to the second tubule through the gel is 200 μm or less, for example 100 μm or less, for example 50 μm or less, for example 10 μm or less, for example 1 μm or less.

37. The apparatus of any one of claims 24 to 36, wherein the microfluidic network comprises a second capillary pressure barrier, defining a boundary between the first region and a third region of the microfluidic network, or a boundary between the second region and a third region of the microfluidic network.

38. The apparatus of claim 37, wherein a gel is provided in the third region of the microfluidic network and confined to the third region by the second capillary pressure barrier;

39. The apparatus of any one of claims 24 to 38, wherein the gel comprises one or more cells or types of cells, e.g. immune cells or cells of mesenchymal origin.

40. The apparatus of any one of claims 24 to 39, wherein one or more types of immune cells, for example T cells, monocytes, macrophages, dendritic cells, and B cells are provided in the lumen and/or the gel so that the one or more immune cells adhere to the first surface of the gel or, when present, the first tubule, or are provided in the gel structure.

41. The apparatus of any one of claims 24 to 40, wherein the microfluidic network comprises at least three inlets, preferably at least four inlets, and wherein at least two inlets are connected to the first region and wherein one or two inlets are connected to the second region.

42. Use of a lumenized gel structure as produced by the method of any one of claims 1 to 23 or use of an apparatus as defined in any one of claims 24 to 39 in an assay, for example an assay selected from one or more of: a barrier function assay, a trans-epithelial electrical resistance (TEER) assay, an immune cell adhesion assay, an immune cell transmigration assay, a transporter assay, and a vasodilation or vasoconstriction assay.
